(19) United States
(12) Patent Application Publication
(10) Pub. No.: US 2010/0120915 A1
(43) Pub. Date: May 13, 2010

(54) Antimicrobials and related methods

(76) Inventor: Johh W. (Jack) Beierle, Upland, CA (US)

Correspondence Address: Lynn G. Foster, 632 12th Avenue, Salt Lake City, UT, 84103, US

(21) Appl. No.: 12/587,740

(22) Filed: Oct. 13, 2009

Related U.S. Application Data
(63) Continuation of application No. 11/712,002, filed on Feb. 28, 2007, now abandoned.
(60) Provisional application No. 60/777,385, filed on Feb. 28, 2006.

(51) Int. Cl. A01N 37/44; A01P 1/00; A01P 3/00

(52) U.S. Cl. 514/561

(57) ABSTRACT

Compositions for antimicrobial, antibacterial, antiviral, fungicidal and sporicidal applications comprise a mixture of alkyl betaine and alkyl amine oxide components together with a protonating agent. The compositions are particularly effective in the treatment and elimination of microorganisms in planktonic cell form as well as in sessile cell form in biofilms. The compositions may be applied in the form of sprays and foams as well as in liquid forms, as a solution or as a balm, as the sole active ingredient or with other active ingredients together with carriers or diluents.

### CONTINUITY

The benefit of U.S. Provisional Application No. 60/777,385, filed Feb. 28, 2006, is claimed:

### BACKGROUND OF THE INVENTION AND RELATED ART

The present invention relates to compositions and methods of using the compositions for antimicrobial, antibacterial, antiviral, fungicidal and sporicidal applications. The compositions and methods are particularly effective in the treatment and elimination of microorganisms in planktonic cell form as well as in sessile cell form in biofilms. The compositions and methods are useful in the treatment of humans and animals as well as inanimate objects, devices and facilities as an antimicrobial sterilant and/or disinfectant.

Medical instruments are typically sterilized or disinfected by introducing them into high temperature and high-pressure autoclaves. Although effective in killing microorganisms, the autoclaves have several significant disadvantages. Autoclaves are typically expensive and have high maintenance costs due to the operating conditions. The extreme pressure and temperature conditions in autoclaves preclude their use in connection with many medical instruments that are sensitive to such extreme environments. Further, autoclaves typically require long cycle periods which range from several minutes to several hours, or even days.

The use of ethylene oxide gas in sealed sterilization chambers at elevated pressures has provided an alternative to autoclaves. These techniques are characterized by long cycle times requiring long exposure times in vacuum and subsequent aeration cycles. Moreover, ethylene oxide is not effective in respect to many medical devices, and it is extremely toxic.

The problems of sterilization become substantially more difficult when biofilms are encountered. Biofilms provide a protective environment for microorganisms existing therein. The organization, protective mechanisms, and cooperation of the various species residing within the biofilms are recognized. Dental plaque, a common biofilm, has been found to contain more than 500 types of microorganisms including bacteria, fungi, viral, spores and even amoebas.

Biofilms are ubiquitous. They are found in a wide range of animal and plant environments as well as inanimate environments such as medical equipment and apparatus, especially where liquids are available to provide a source of nutrients. In all cases, the extra-cellular matrix of the biofilm secures the microorganisms together and to a recipient surface. The matrix also serves to provide protection since a substance will have a difficult time diffusing into the center of the matrix if it reacts with the cells or the matrix material it encounters along the way. In turn, environmental changes result within the matrix and a variety of chemical environments arise with corresponding differences in the cells, even though they are genetically identical, there are changes in genetic expression and phenotypic changes.

### SUMMARY OF THE INVENTION

Applicant has now discovered that selected compositions are effective in the control and elimination of microorganisms in planktonic cell form as well as in sessile cell form in biofilms. The compositions are effective for antimicrobial, antibacterial, antiviral, fungicidal and sporicidal treatments. The compositions are substantially nontoxic and otherwise do not harm or damage animal tissue or cells. The compositions are particularly useful as sterilants/disinfectants at room temperature and with relatively short treatment times and dilute concentrations.

Sterilization is defined as the complete killing of all foreign organisms. Herein, sterilization is deemed to be indicated by the inactivation (or killing) of a significant challenge (e.g., one million cfu) of Bacillus stearothermophilus spores at ambient or room temperature conditions, upon contact with an effective amount of the compositions of the present invention. If a process is successful in inactivating a significant challenge of B. stearothermophilus spores, then it is recognized that all pathogenic bacterial spores, as well as viruses, fungi, and vegetative bacteria exposed to those conditions at that time, are also inactivated. Editor, Joseph M. Ascenszi, Handbook of Disinfectants and Antiseptics, Marcel Dekker, Inc., 1996.

Disinfection is understood to be the selective elimination of selected undesirable microorganisms to prevent their transmission, i.e., the reduction of the number of infectious organisms to a value below that necessary to cause infection. Antisepsis is the application of an antimicrobial to skin or other living tissue to inhibit the growth of and/or destroy microorganisms.

The effectiveness of the compositions in the control and killing of biofilms is most surprising. It is believed that the compositions themselves disrupt the physical state of the biofilm to gain better access to the sessile cells therein and enhance the antimicrobial, antibacterial, antiviral, fungicidal and sporicidal effects of the compositions per se on the sessile cells after adoption of the biofilm phenotype.

The active ingredients or components of the compositions comprise a mixture of alkyl betaine and alkyl amine oxide components together with a protonating agent. The mixture may be formed by combining the betaine and amine oxide components, and than adding the protonating agent or acid together with a suitable solvent to provide the overall resulting mixture. The concentration of the active betaine and amine oxide ingredients may range from about 0.01 part to about 40 parts by weight per 100 parts total, and, more preferably, from about 0.02 part to about 20 parts. As used herein, the reference to “part” or “parts” is by weight based on 100 parts total of the mixture of the composition discussed unless otherwise indicated by the context.

The effective ingredients of the inventive compositions comprise in admixture:
(a) a mixture of two alkyl-N-betaines,
(b) an alkyl-N,N-dimethylamine oxide, and
(c) a protonating agent, such as hydrochloric acid, acetic acid or citric acid, in an amount sufficient to adjust the pH of the overall composition in the range of from about 4 to about 7.5.

Each of the betaines and the amine oxide is present in an amount ranging from about 0.01 part to about 20 parts, and more preferably, from about 0.02 part to about 10 parts.

The betaine compositions are:

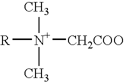

where R is a mixture of higher alkyl having from 12 to 14 carbon atoms. Illustrative of such mixtures are lauryl-N-betaine and myristyl-N-betaine in lauryl/myristyl mixture ratios of from about 30:70 to about 70:30. More preferably, the mixture ratio is from about 60:40 to about 50:50.
The amine composition is:

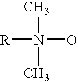

where R is a higher alkyl containing 16 carbon atoms, in the form of a cetyl radical. Accordingly, the amine composition comprises cetyl-N,N-dimethylamine oxide.

The use of a protonating agent supplies the required pH and cooperates in the effectiveness of the compositions and processes herein. Illustrative protonating agents include any suitable organic or inorganic acid, such as hydrochloric acid, phosphoric acid, sulfuric acid, citric acid, acetic acid, nicotinic acid, and the like. The solution may have a pH in the range of from about 4 to about 7.5, and more preferably, from about 4 to about 5, and most preferably, about 4.85. The protonating agent is contained in a suitable solvent such as water or a suitable lower alcohol, C1 to C4 aliphatic alcohol, or combinations thereof. With the use of buffers, effective kill is achieved at pH values in the range of from about 6 to about 7.4. The pH may be lowered with the use of HCl and increased with the use of phosphate buffered saline.

In accordance with the invention, the compositions and methods have utility in sterilant applications at room temperature and atmospheric pressure, and also at elevated temperatures and pressures, with direct application of the sterilant to the article to be sterilized. The compositions and methods are particularly useful in health care facilities as well as field environments. Similarly, the compositions and methods may be used in industrial applications, especially those involving water supply or processing.

The compositions are useful as sterilants for application to medical implements, especially as may be encountered in military uses under field conditions. In this respect, the sterilant also acts as a cleaner or disinfectant, or a component thereof. The compositions are safe for application to human tissue and for human ingestion.

The compositions have antimicrobial properties including a high-level antimicrobial kill of fungi, gram positive and gram negative bacteria and spore forming microbes. Therapeutic and prophylactic effectiveness has been confirmed in connection with a variety of activities described hereinafter. And, as noted above, the compositions are effective against planktonic cell and sessile cell forms as well as a biofilm combatant including penetration, dislodgement and/or disintegration of the biofilm structure.

The compositions of the present invention are surfactant in nature including hydrophobic molecule ends. The betaines are recognized as amphoteric surfactants. The surfactant characteristics also cause the compositions to display a tendency to foam in the air when mixed in a liquid dispensing action such as discharge from a pump container. The resulting foam will be maintained for less than about a minute under ambient conditions, room temperature and atmospheric pressure. Thereafter, the foam collapses to form a continuous film. The film has a tendency to be retained on the supporting substrate such as inorganic metal or glass surfaces and organic surfaces such as human skin. The composition is therefore useful to form a “liquid bandage.” The resulting film provides prophylactic-type protection in the nature of a barrier as well as antimicrobial, antibacterial, antiviral, fungicidal and sporicidal effects.

The inventive compositions are also useful in connection with devices requiring a relatively contamination free or disinfected or sterile environment for frictionally engaged moving parts. In such an environment, the compositions have been found to act as a lubricant as well as a sterilant/disinfectant. For example, medical instruments such as dental hand pieces.

### BRIEF DESCRIPTION OF THE DRAWINGS

FIG. 1 is a graph showing Pseudomonas aeruginosa kill rate over time following treatment with the compositions of the invention and following treatment with several comparative compositions;

FIG. 2 is a graph showing Candida albicans kill rate over time following treatment with the compositions of the invention and following treatment with several comparative compositions;

FIG. 3 is a graph showing E. coli kill rate over time following treatment with the compositions of the invention and following treatment with several comparative compositions;

FIG. 4 is a graph showing Bacillus stearothermophilus kill rate over time following treatment with the compositions of the invention and following treatment with several comparative compositions;

FIG. 5 is a graph showing Candida albicans kill rate over time following treatment with the compositions of the invention at reduced concentrations;

FIG. 6 is a graph showing E. coli kill rate over time following treatment with the compositions of the invention at reduced concentrations;

FIG. 7 is a graph showing Pseudomonas aeruginosa kill rate over time following treatment with the compositions of the invention at reduced concentrations;

FIG. 8 is a graph showing Bacillus stearothermophilus kill rate over time following treatment with the compositions of the invention at reduced concentrations;

FIG. 9 is a graph showing mixed oral flora kill rate over time following treatment with the compositions of the invention at reduced concentrations;

FIG. 10 is a graph showing Methicillin-Resistant Staphylococcus aureus (MRSA) kill rate over time following treatment with the compositions of the invention;

FIG. 11 is a graph showing Staphylococcus aureus kill rate over time following treatment with the compositions of the invention;

FIG. 12 is a graph showing Acinetobacter baumannii kill rate over time following treatment with the compositions of the invention;

FIG. 13 is a graph showing Vancomycin-resistant Enterococci (VRE) kill rate over time following treatment with the compositions of the invention;

FIG. 14 is a graph showing the kill rates of Streptococcus pyogenes versus the compositions of the invention and various commercially available disinfectants;

FIGS. 15 through 26 are photomicrographs showing various biofilms treated with the compositions of the invention;

FIG. 27 is a graph reporting a survey count of the microbes, molds and Beta hemolytic pathogens present in untreated areas of a dental facility;

FIG. 28 is a graph similar to FIG. 28 reporting the count of the microbes, molds and Beta hemolytic pathogens after five minutes following treatment with the composition of the invention;

FIG. 29 is a graph similar to FIG. 29 reporting the count of the microbes, molds and Beta hemolytic pathogens after one minute following a spray treatment with the composition of the invention;

FIG. 30 is a table presenting data concerning colony forming units in respect to the treatment of raw salmon and chicken with Formula 5 incubated at room temperature and 37° C.;

FIG. 31 is a graph presenting data concerning colony forming units in respect to the treatment of raw salmon with Formula 5 incubated at room temperature and 37° C.; and

FIG. 32 is a graph presenting data concerning colony forming units in respect to the treatment of raw chicken with Formula 5 incubated at room temperature and 37° C.

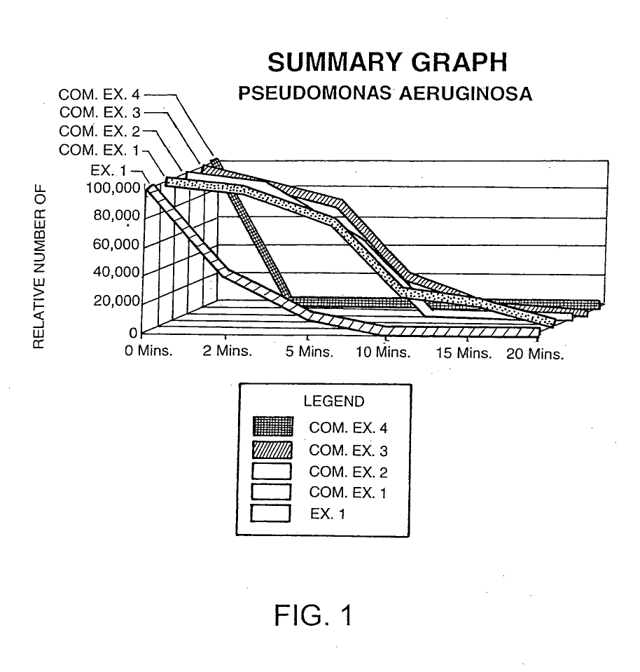

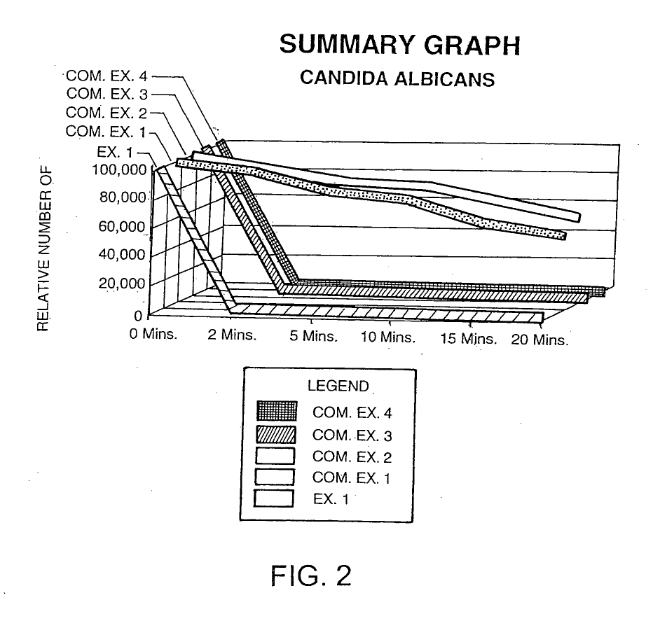

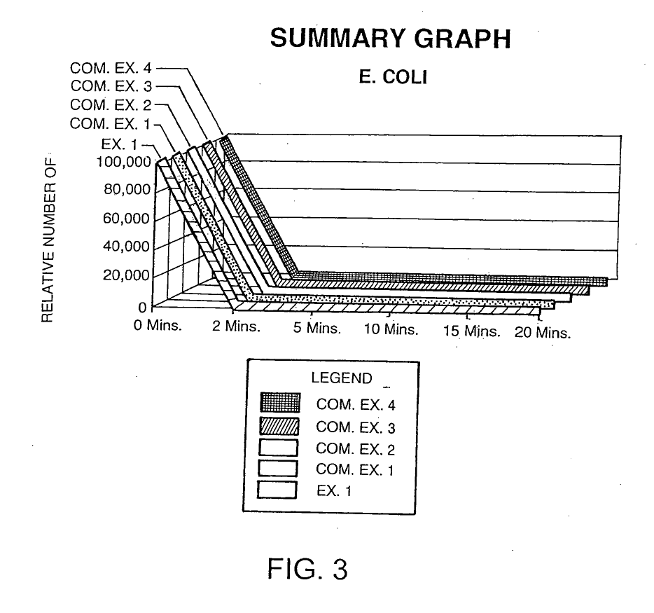

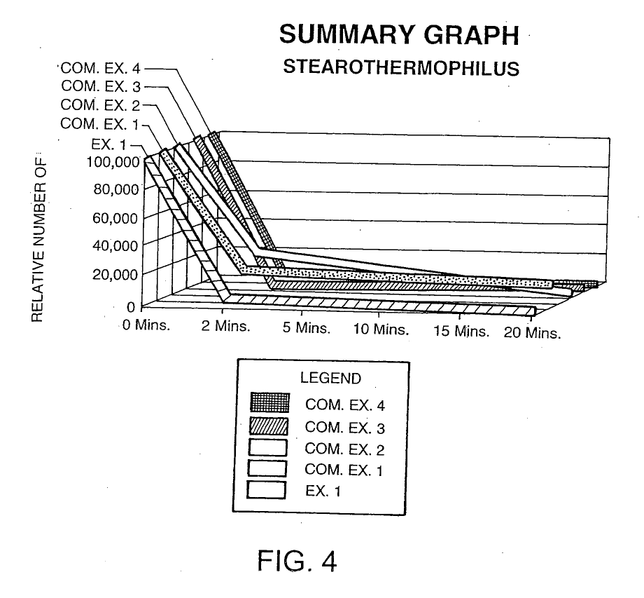

### DETAILED DESCRIPTION

The compositions may be applied or administered in conventional manners in aerosol or foam forms as well as in liquid form, as a solution or as a balm, as the sole active ingredient or with other active ingredients together with carriers or diluents as are known in the art. The compositions and methods of the present invention are initially described herein with respect to their sterilant applications and sterilizing utilities.

In the following examples and comparative examples, the components are reported in weight percent based on the total weight or the numerically corresponding parts per 100 parts total, unless otherwise indicated by the text or context of the discussion. Distilled water is used as the solvent in all of the examples to form the overall mixture or to prepare dilutions thereof.

### Example 1

The ingredients of the composition or Example 1 comprise in admixture:
(a) Lauryldimethylbetaine, 0.84% by weight,
(b) Myristyldimethylbetaine, 0.36% by weight,
(c) Cetyldimethyl amine oxide, 1.20% by weight, and
(d) Citric acid in an amount sufficient to adjust the pH of the overall composition to about 4.85.

The betaine and amine oxide active ingredients of the composition may be combined at room temperature with mixing. The acid may be combined with the foregoing ingredients or subsequently combined together with distilled water.

### Comparative Example 1

Comparative Example 1 is prepared using the same procedures as described above and comprises in admixture:

(a) Cetyldimethylbetaine 1.2% by weight,

(b) Myristyldimethyl amine oxide 1.2% by weight,

(c) Citric acid in an amount sufficient to adjust the

pH of the overall composition to about 4.85.

### Comparative Example 2

Comparative Example 2 is prepared using the same procedures as described above and comprises in admixture:

(a) Lauryldimethylbetaine 1.26%,

(b) Myristyldimethylbetaine 0.54%,

(c) Cocoamidopropyl amine oxide 1.80%, and

(d) Citric acid in an amount sufficient to adjust the

pH of the overall composition to about 4.85.

### Comparative Example 3

Comparative Example 3 is prepared using the same procedures as described above and comprises in admixture:

(a) Lauryldimethylbetaine 1.26%,

(b) Myristyldimethylbetaine 0.54%,

(c) Myristyl-bis(2-hydroxyethyl)amine oxide 1.26%,

(d) Cetyl-bis(2-hydroxyethyl)amine oxide 0.54%, and

(e) Citric acid in an amount sufficient to adjust the

pH of the overall composition to about 4.85.

### Comparative Example 4

Comparative Example 4 is prepared using the same procedures as described above and comprises in admixture:

(a) Cetyldimethylbetaine 1.21%,

(b) Lauryldimethylbetaine 0.85%,

(c) Myristyldimethylbetaine 0.36%,

(d) Myristyldimethyl amine oxide, 1.70%,

(e) Cetyldimethyl amine oxide 0.73%, and

(f) Citric acid in an amount sufficient to adjust the

pH of the overall composition to about 4.85.

### Comparative Example 5

Comparative Example 5 is prepared using the same procedures as described above and comprises in admixture:
(a) Lauryldimethylbetaine 2.43%,
(b) Cetyldimethyl amine oxide 2.94%,
(c) Citric acid in an amount sufficient to adjust the pH of the overall composition to about 4.85.

### Comparative Example 6

Comparative Example 6 is prepared using the same procedures as described above and comprises in admixture:

(a) Myristyldimethylbetaine 2.43%,

(b) Cetyldimethyl amine oxide 2.94%,

(c) Citric acid in an amount sufficient to adjust the

pH of the overall composition to about 4.85.

### Comparative Example 7

Comparative Example 7 is prepared using the same procedures as described above and comprises in admixture:

(a) Cocodimethylbetaine 2.49%,

(b) Cocodimethyl amine oxide 2.49%,

(c) Citric acid in an amount sufficient to adjust the

pH of the overall composition to about 4.85.

The Cocodimethylbetaine is a commercially available blend of alkyl substituted betaines with the following approximate compositions of alkyl components by weight percent:

| | | |
|---|---|---|
| C⁸ - 3.2% | C¹⁰ - 6.3% | C¹² - 51.9% |
| C¹⁴ - 20.7% | C¹⁶ - 12.1% | C¹⁸ - 5.9% |

The “coco” species is economically favored in many other applications, but not found particularly useful herein.

### Comparative Example 8

Comparative Example 8 is prepared using the same procedures as described above and comprises in admixture:
(a) Cocodimethylbetaine 2.49%,
(b) Cetyldimethyl amine oxide 2.49%,
(c) Citric acid in an amount sufficient to adjust the pH of the overall composition to about 4.85.

The “coco” species is again used in this comparative example.

The kill rates of the compositions Example 1 and Comparative Examples 1-4 were determined with respect to Pseudomonas aeruginosa, Candida albicans, E. coli, and Bacillus stearothermophilus. The kill rate of each of the compositions was determined by combining a 200 microliter dilution of the composition being tested with a 2 ml sample of bacteria containing two billion colony forming units (cfu's). It should be appreciated that conventional testing may be against several million cfu's. The mixture was maintained at 70° F., and 0.5 ml aliquots were withdrawn at various time points. The aliquots were plated-out using standard plate count methodology to determine the reduction of cfu's per time point.

The kill rates of the compositions of Comparative Examples 5-8 were determined with respect to E. coli using the above procedures and an initial bacteria sample containing one billion colony forming units.

| | | | | | |
|---|---|---|---|---|---|
| | % | | | | |
| EXAMPLE | ACTIVE | PSEUDOMOAS | CANDIDA | | BACILLUS |
| NO. | INGRED. | AERUGINOSA | ALBICANS | E. COLI | STEAROTHERMOPHILUS |

MINUTES TO COMPLETE KILL

| | | | | | |
|---|---|---|---|---|---|
| Ex. 1 | 2.40 | 5 | 2 | 2 | 2 |
| Com. Ex. 1 | 2.40 | 20 | Fail | 2 | 20+ |
| Com. Ex. 2 | 3.60 | 20 | Fail | 2 | 20+ |
| Com. Ex. 3 | 3.60 | 12 | 2 | 2 | 3 |
| Com. Ex. 4 | 4.85 | 2 | 2 | 2 | 2 |
| Com. Ex. 5 | 5.37 | | | 15 | |
| Com. Ex. 6 | 5.37 | | | 10 | |
| Com. Ex. 7 | 4.98 | | | 10 | |
| Com. Ex. 8 | 4.98 | | | 10 | |

As indicated, the composition of Example 1 effectively kills the indicated organisms during relatively short exposure or contact times in the order of seconds or minutes. Moreover, the results are achieved at a lower concentration of active ingredients as compared with the compositions of Comparative Examples 3 and 4. The efficacy of the composition of Example 1 in the “kill” and limitation of growth of a panel of bacteria shows Example 1 to be a broad spectrum efficient anti-microbial agent.

### Example 2

The ingredients of the composition of Example 2 comprise in admixture:
(a) Lauryldimethylbetaine, 1.95 parts by weight,
(b) Myristyldimethylbetaine, 1.05 parts by weight,
(c) Cetyl dimethyl amine oxide, 3.00 parts by weight; and
(d) citric acid in an amount sufficient to adjust the pH of the overall composition to about 4.85.

The betaine and amine oxide active ingredients of the composition may be combined at room temperature with mixing. The acid may be combined with the foregoing ingredients or subsequently combined together with distilled water. The resulting composition contains 5.00% active ingredients based on the weight of the betaine and amine oxide components, and for purposes herein, it is considered to be a 1:5 dilution used to make further dilutions as reported below.

### Example 3

The composition or Example 2 was further diluted using distilled water to provide a 1:10 dilution to prepare Example 3. Example 3 has a concentration or active ingredients, the total betaine and amine oxide components, equal to 2.50% by weight.

### Example 4

The composition of Example 2 was further diluted using distilled water to provide a 1:60 dilution ratio to prepare Example 4. Example 4 has a concentration of active ingredients, the total betaine and amine oxide components, equal to 0.41% by weight.

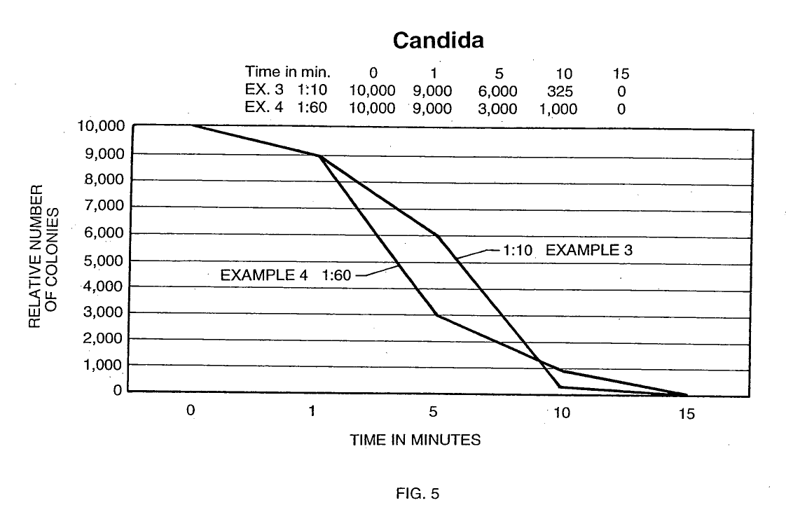

The effectiveness of kill or Examples 3 and 4 were measured against Candida albicans at room temperature beginning with an initial microbe count of one billion. The results are shown in FIG. 5. The initial kill rate over the first minute was similar for Examples 3 and 4. Thereafter, the more concentrated solution of Example 3 exceeded Example 4. However, both concentrations provided substantially 100% kill by 15 minutes.

These examples demonstrate that the compositions of the present invention are exceptionally effective against Candida albicans, one of the most difficult microbes to kill. Candida albicans is a member of the fungal family; primarily a yeast, but a dimorphic microbe, capable of developing a mold-like appearance under proper environmental conditions.

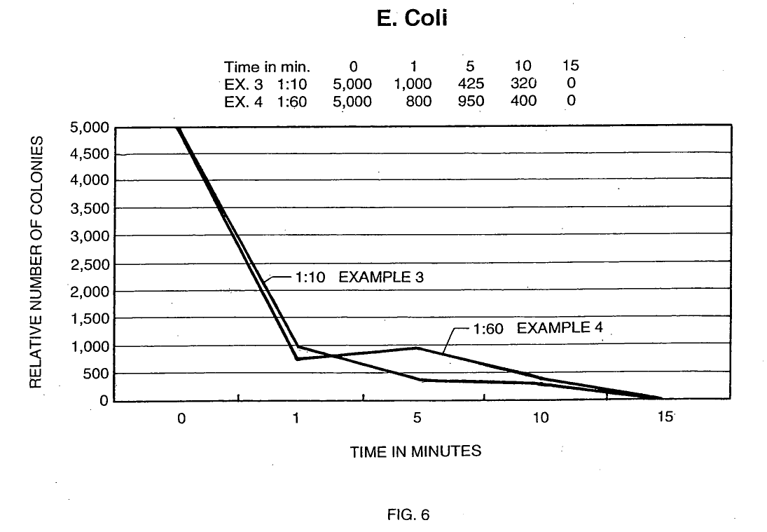

Referring to FIG. 6, the kill rate or Examples 3 and 4 against E. coli is reported. Again, at time zero, there were about one billion microbes present, and equivalence or kill is seen with substantially 100% kill being achieved after 15 minutes at room temperature.

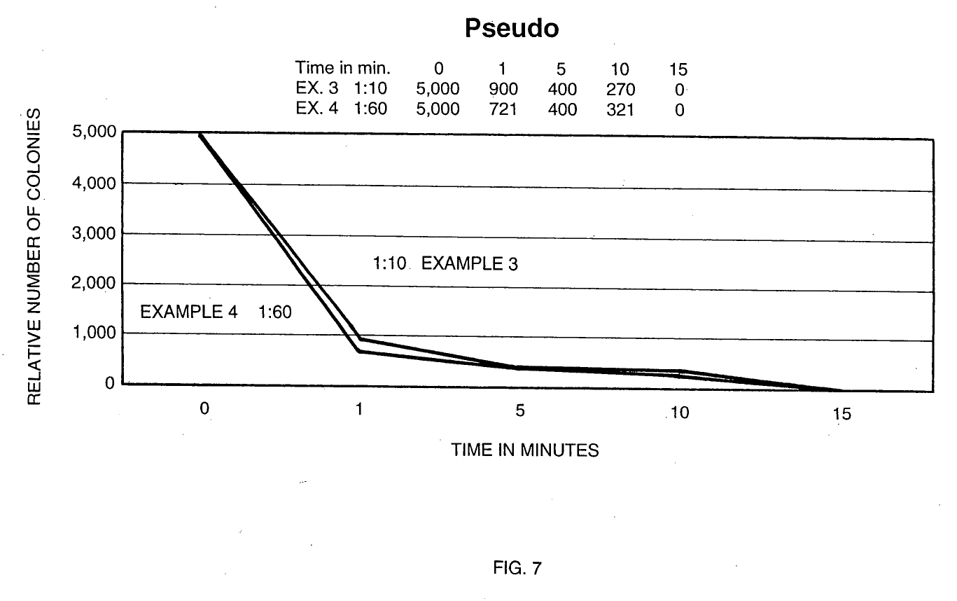

The kill rates of the compositions of Examples 3 and 4 against Pseudomonas aeruginosa are reported in FIG. 7. Again, equivalence or kill is seen with total kill by 15 minutes.

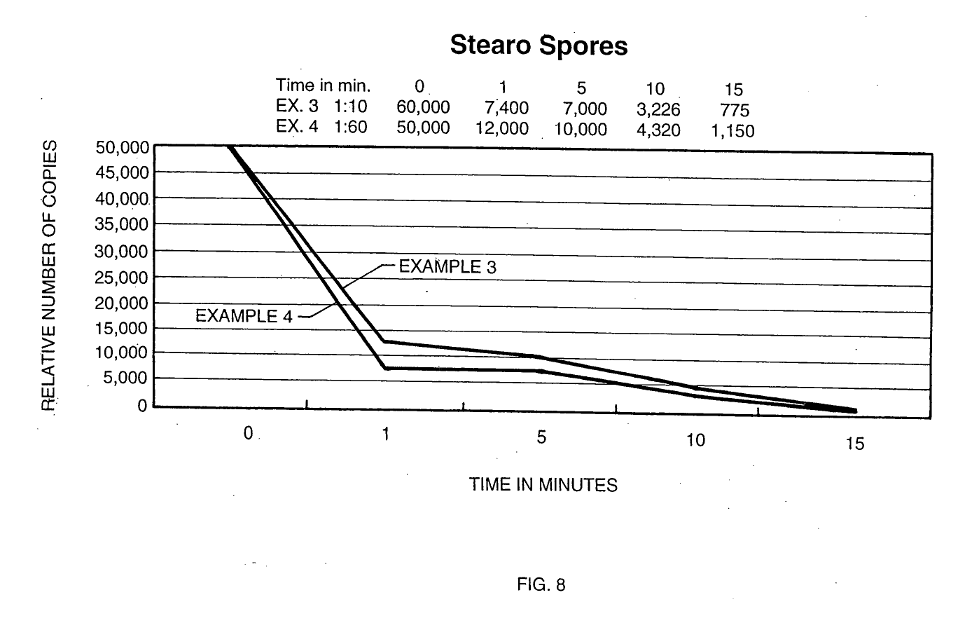

Referring to FIG. 8, the effectiveness or kill of Examples 3 and 4 against Bacillus stearothermophilus is reported. As shown, substantial kill is achieved in about 1 minute and substantially complete kill occurs in 15 minutes.

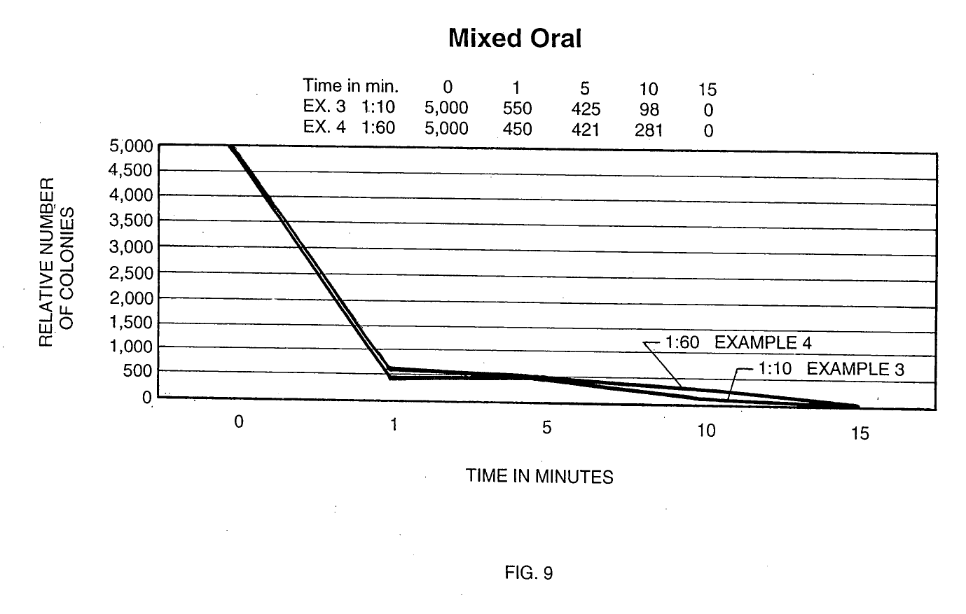

The effectiveness or kill rates of Examples 3 and 4 with respect to biofilms in the form of oral flora is reported in FIG. 9. The oral cavity is known to contain In excess of 500 species. This complex milieu containing clusters of plaque biofilm microbes takes about a minute to achieve a 99% kill and up to 10 minutes for complete eradication. However, there is again a general equivalence of effectiveness with substantial kill occurring in one minute and complete kill occurring in 15 minutes.

The compositions of the present invention have also been evaluated against Methicillin-Resistant Staphylococcus aureus (MRSA). MRSA is a type of Staphylococcus aureus that is resistant to antibiotics called beta-lactams. Betalactam antibiotics include Methicillin and other more common antibiotics such as oxacillin, penicillin and amoxicillin. Staph infections, including MRSA, are most frequently found among persons in hospitals and health care facilities, such persons having weakened immune systems. These Healthcare-associated staph infections include surgical wound infections, urinary tract infections, bloodstream infections, and pneumonia. However, staph and MRSA infections can also cause illness in persons outside of hospitals and health care facilities. (See CDC MRSA Public Info.)

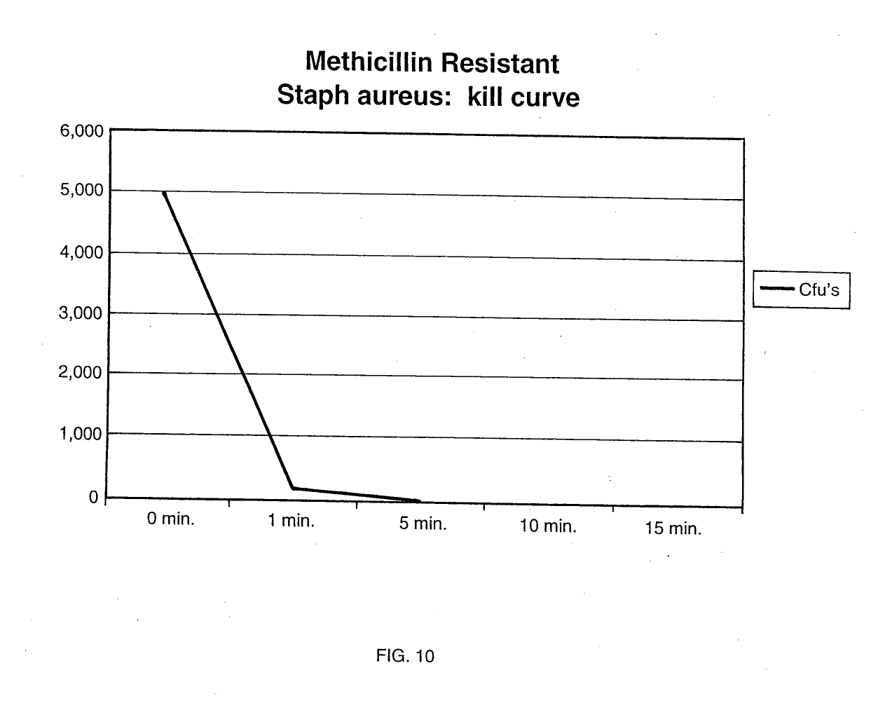

Using the same procedures as described above, the composition or Example 2, diluted with distilled water to a of 1:20 dilution, is combined with a one billion cfu sample of MRSA at room temperature. The MRSA kill rate over time is reported in the graph of FIG. 10. As shown, substantial kill occurs in about one minute and substantially complete kill occurs in less then about 8 minutes with a concentration of about 1.25% by weight.

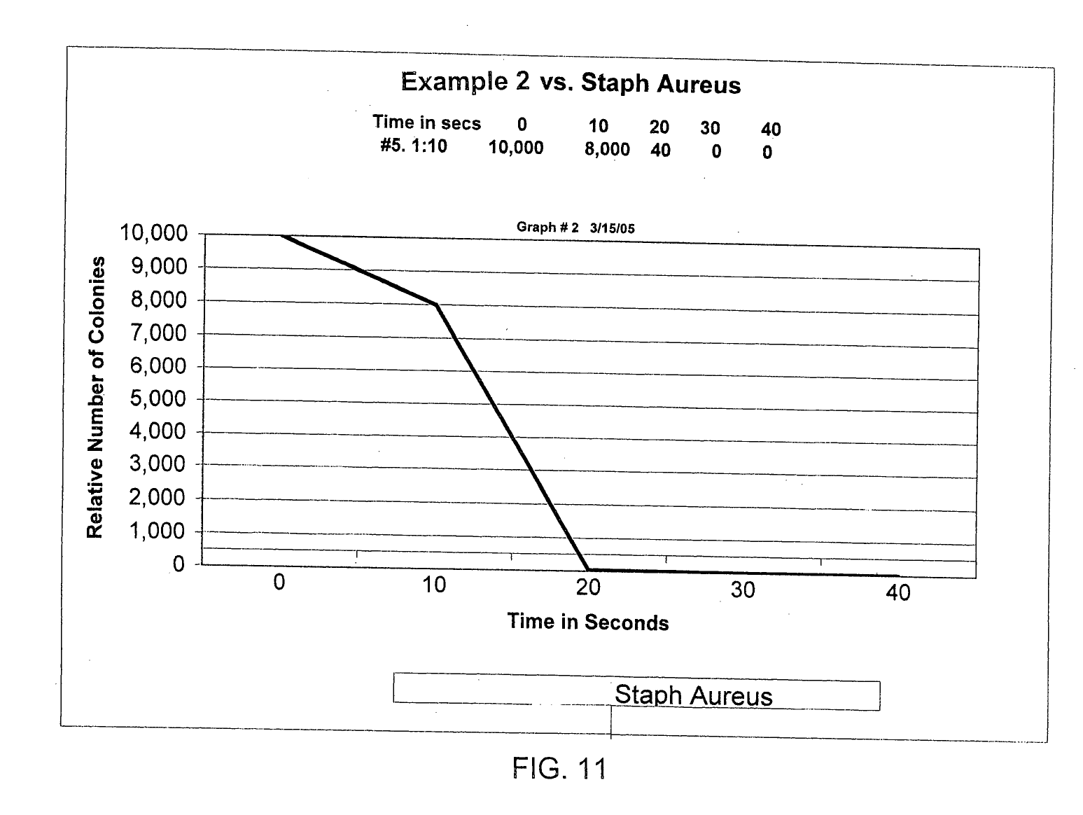

The compositions of the present invention have also been evaluated against Staphylococcus aureus to demonstrate the rapid kill achieved. Using the same procedures as described above, the composition of Example 2, diluted with distilled water to a 1:10 dilution, is combined with a one billion cfu sample of Staph aureus at room temperature. The Staff aureus kill rate over time is reported in the graph of FIG. 11. (In FIG. 11, the scale is arbitrarily set for a 10,000 cfu start to demonstrate reduction even though a one billion cfu sample is present at time zero.) As shown, substantial kill occurs in about ten seconds and substantially complete kill occurs in less then about 30 seconds with a concentration of about 1.25% by weight.

The compositions of the present invention have also been evaluated against Acinetobacter baumannii which is a species of gram-negative bacteria commonly found in water and soil. During 1963-2003, A. baumannii became an increasingly important cause of nosocomial infections, particularly in ICU's. Treatment of infections attributed to A. baumannii can be difficult because the organism has intrinsic resistance to certain antimicrobial agents and has acquired resistance to many others. An increasing number of A. baumannii bloodstream infections in patients in military medical facilities involving service members injured in the Iraq/Kuwait region has been observed. The number of these infections and their resistance to multiple antimicrobial agents underscore the importance of infection control during treatment in combat and health-care settings, and the need to develop new antimicrobial drugs to treat these infections. CDC, MMWR, Weekly, Nov. 19, 2004/53 (45) 1063-1066.

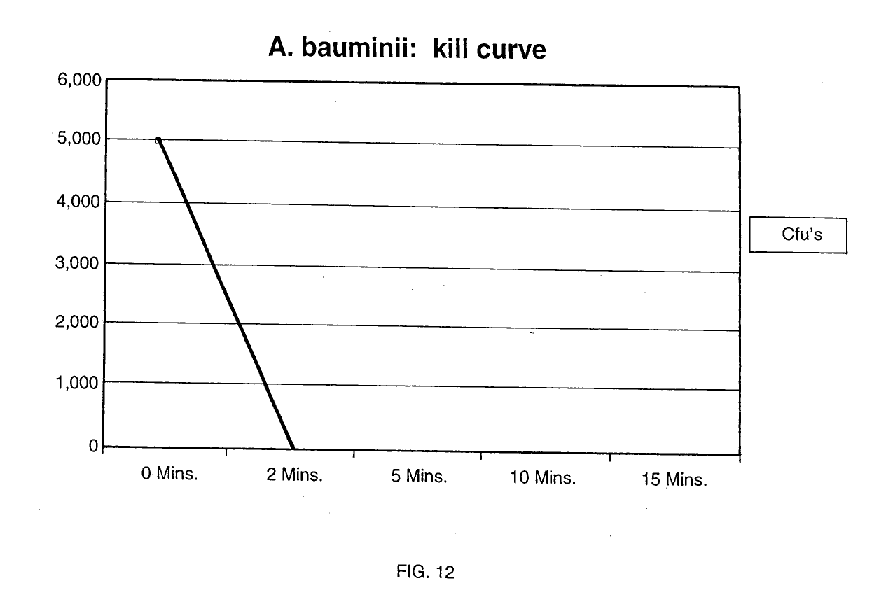

Using the same procedures as described above, the composition of Example 2, diluted with distilled water to a 1:40 dilution, is combined with a one billion cfu sample of A. baumannii at room temperature. The A. baumannii kill rate over time is reported in the graph of FIG. 12. As shown, substantial kill occurs in about one minute and substantially complete kill occurs in less then about 3 minutes with a compositions concentration of about 0.63% by weight.

The compositions of the present invention have also been evaluated against Vancomycin-resistant Enterococci (VRE). There are two types of Vancomycin resistance, namely, inherent and acquired. It is believed that Enterococci can become resistant to vancomycin by acquisition of genetic information from another organism. Rice, Emerging Infective Diseases, Vol. 7, No. 2, March-April 2001.

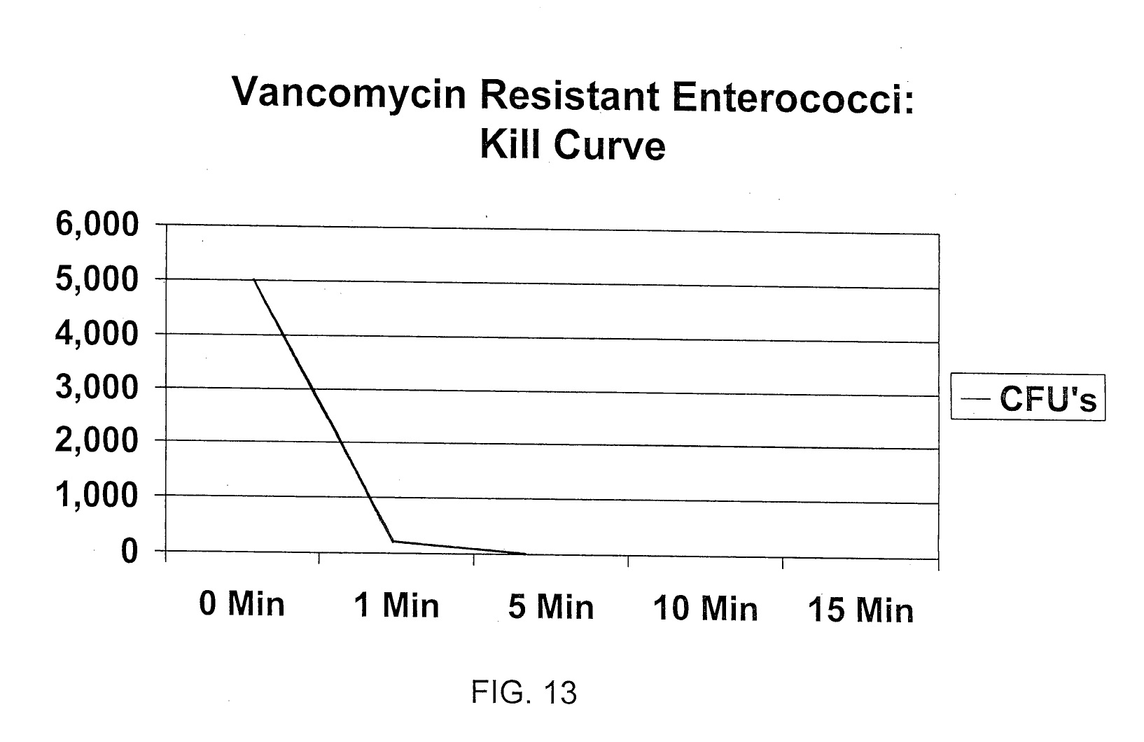

Using the same procedures as described above, the composition of Example 2, diluted with distilled water to a 1:20 dilution, is combined with a one billion CFU sample of VRE at room temperature. The VRE kill rate over time is reported in the graph of FIG. 13. As shown, substantial kill occurs in about one minute and substantially complete kill occurs in less then about 3 minutes with a concentration of about 1.25% by weight.

The compositions of the present invention are useful as disinfectants, such as Betadyne antiseptic and microbiocidal, and may be used in similar manners. In addition to Betadyne, commercially available disinfectants used in health care facilities include Vitaphene, Povidone Iodide, Aerocide, Cidex and Sporocidium.

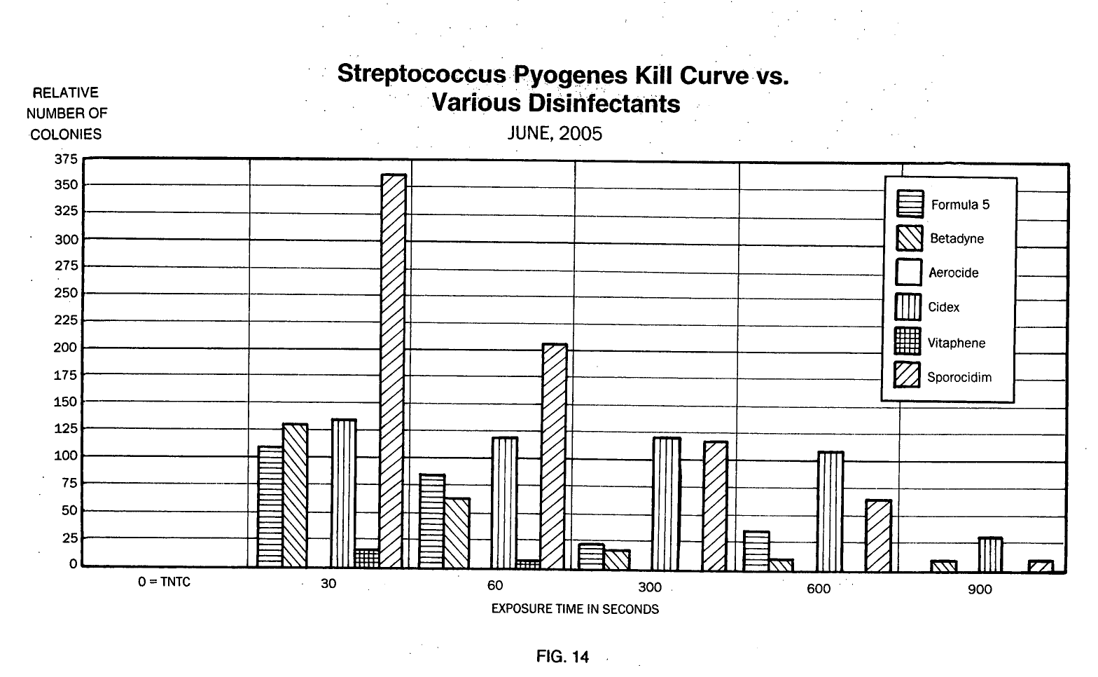

A comparison of the effectiveness of each of the foregoing against Streptococcus pyogenes is reported in FIG. 14. These disinfectants are comparatively evaluated herein at their commercially supplied concentrations. In each case, a 2 ml dose of 10⁸ Streptococcus/ml was tested against 200 microliters of the disinfectant. As shown, time points were measured in seconds up to 900 seconds. In all instances, Example 2 was as good, if not better, than the other disinfectants.

The disinfectants were also tested against mixed oral bacteria. The same dosage as described above was prepared of the oral bacteria and it was tested against a 200 micrometer sample of the disinfectant. In this instance, only Betadyne and Vitaphene approach the effectiveness of the composition of Example 2. The results are reported in the following Table 2.

TABLE 2

| 0 Sec. | 30 Sec. | 60 Sec. | 5 Min. | 10 Min. | 15 Min | |
|---|---|---|---|---|---|---|
| TNTC | 0 | 0 | 0 | 0 | 0 | Example 2 |
| TNTC | 15 | 0 | 0 | 0 | 0 | Betadyne¹ |
| TNTC | 8,000 | 750 | 605 | 550 | 470 | Aerocide² |
| TNTC | TNTC | 9,000 | 8,000 | 7,200 | 1,275 | Cidex³ |
| TNTC | 25 | 3 | 0 | 0 | 0 | Vitaphene⁴ |
| TNTC | TNTC | TNTC | TNTC | TNTC | 8,000 | Sporocidium⁵ |

¹Betadyne a 10% iodide solution by Purdue Products L.P.
²Aerocide an o-phenylphenol 0.10%, 4-chloro-2 cyclopentylphenol 0.08%, lauric diethanolamide 0.20% and triethanolamine dodecyl benzenesulfonate 0.33% by GF Health Products, Inc.
³Cidex a 2.4% gluteraldehyde by Johnson and Johnson Div. of Ethicon of Irving, CA.
⁴Vitaphene a 9.0% alpha phenylphenol and 1.0% o-phenylphenol by Block Drug Corporation, Jersey City, NJ.
⁵Sporocidium a 1.56% phenol, 0.06% sodium phenate by Sporicidin International, Rockville, MD.
TNTC means “too numerous to count”.

The compositions of the present invention are also useful for infection control in the antiseptic care of incisional and burn wounds. Wound contamination and the subsequent decontamination of wounds is of interest in a combat care setting. A number of methods are currently in use in wound and instrument decontamination including sterilization, disinfection, and antisepsis.

Contamination is defined as the introduction of microorganisms into tissues or other materials, whereas decontamination is defined as the reverse. That is, disinfection or sterilization of infected wounds to an acceptable level (noninfectious level).

The efficacy of the compositions of the invention against human pathogenic bacteria was evaluated. For this purpose, evaluation of the bacterial “kill” on uncompromised normal skin was evaluated. In these experiments, bacterial strains of Staphylococcus aureus. Pseudomonas aeruginosa and normal oral flora were introduced to the shaved backs of rabbits in concentrations of 1×10⁹ cfu/25 ul. Following application of the bacterial treatments, a saline control, Betadyne and the composition of Example 2 were applied at a rate of 100 ul per square inch. Betadyne and Example 2 were found to prevent bacterial growth, that is, they showed similar results in the limiting of the growth of the applied bacteria and ultimately killing the bacteria.

The antiseptic effect of treatment with saline, Betadyne and Example 2 in a partial thickness incision model was also evaluated. A 2.5 cm incision extending through the dermal layer was made in the shaved backs of New Zealand White rabbits. As in the clear skin studies, the various microbes at similar concentrations were placed in the incisions and the site treated with 100 ul of saline, Betadyne or the composition of Example 2. The incisions were covered with occlusive Hilltop chamber dressings. Once again, the Betadyne and the composition of Example 2 showed like inhibition and kill of the bacteria.

In the evaluation of burn wounds, the antiseptic properties of Betadyne and the composition of Example 2 were compared against saline control. In the burn model, the wound is created and the bacteria applied to the healing wound as would be the case in the field. In this instance, the shaved backs of guinea pigs were burned and covered with an occlusive Hilltop chamber dressing for 24 hours. Thereafter, the burn wound is debrided and intentionally infected with bacteria as described above. Once again, the antiseptic properties of Betadyne and Example 2 were comparable.

The composition of Example 2 is as effective as Betadyne in the decontamination of intentionally contaminated clear skin, incisions and partial thickness burns.

Additional microbes of particular interest were evaluated in further rabbit studies. The additional test microbes included:
Methicillin Resistant Staph aureus (MRSA)
Strep pyogenes
Vancomycin Resistant Enterococci faecalis (VRE)
E. coli
Pseudomonas aeruginosa

After the animals were anaesthetized and shaved as previously described above, a deep wound was made on each side of the backs of the animals with a scalpel. One of the wounds was for Betadyne and one wound was for Example 2. Microbial cultures grown on blood agar were inoculated heavily on cotton swabs directly from large colonies and rubbed into the wound sites. Inoculation of the wounds was estimated by examination of comparable cotton swabs which had their contents dislodged by sonication or high-speed circular spin procedures to suspend and isolate bacteria from the cotton swab tip. The microbes isolated in suspension were diluted by 10 fold dilution procedures and counted in pour plates of Trypticase soy agar, yeast extract and Todd-Hewitt broth (10:1:5). The cfu account revealed a range of 100-120 million cfu's/swab. Therefore, a direct inoculation of about 110 million bacteria were swabbed directly into rabbit wounds. Massive inocula were therefore achieved. Distinctive colonies were stained for morphology and gram staining characteristics.

The following day, two milliliter doses of Betadyne and Example 2 were respectively applied at room temperature by dropper at various points. The composition of Example 2 was applied at a 1:10 dilution (2.5% by weight concentration of active ingredients) in two milliliter doses by drop wise application to the wound. Swabs were taken after one minute, five minutes and one hour to determine cfu's remaining on the wound. This was followed by a three-day waiting period with no additional disinfectant applied.

Swabs taken from the animals were placed in 3 ml saline and vortexed for 30 seconds to remove bacteria. Samples were spread by plastic spreaders on blood agar and incubated for 48 hours for cfu analysis. The results are reported below Table 3.

TABLE 3

| | | Strep | | | |
|---|---|---|---|---|---|
| | MRSA | Pyogenes | VRE | E coli | Pseudomonas |
| Baseline | 378 | 217 | 212 | 406 | 397 |
| 1 min. | 335 | 220 | 243 | 356 | 350 |
| Betadyne | | | | | |
| 1 min. | 135 | 117 | 36 | 200 | 300 |
| Example 2 | | | | | |
| 5 min. | 286 | 65 | 60 | 165 | 112 |
| Betadyne | | | | | |
| 5 min. | 36 | 17 | 10 | 40 | 56 |
| Example 2 | | | | | |
| 24 hrs. no | 585 | 305 | 393 | 375 | 428 |
| further | | | | | |
| treatment | | | | | |

As indicated by the data, treatment with Example 2 after one minute shows some microbe reduction. However, there is little effect, if any, for Betadyne. After five minutes, good reduction of all five microbes is found with Example 2. In comparison, fair to good reduction is also found with Betadyne after this passage of time.

After 24 hours, the microbes reestablish themselves indicating that the multiple doses of disinfectants should be applied over the course of several days for wound healing, surgical intervention or other treatment. Multiple applications or continuous contact with the inventive compositions, which are both possible due to its low toxicity level, would keep the wound in an excellent stage for healing and/or subsequent surgery.

The efficacy of the inventive compositions in respect to naturally encountered microbes in various soils was also investigated. To that end, soil samples were taken from the following locations.
Desert—the Mojave Desert, Calif., 60 miles north of the City of Mojave and 20 miles south or Adelanto. Altitude 2,300 feet above sea level.
Mountain—The Sierra Nevada Mountain Range, McGee Canyon, Calif., 13 miles south of the town of Mammoth Lakes at 7,600 feet above sea level.
Beach—A beach on the island of Kauai, Hi., 12 miles north or the airport at sea level elevation.

The collected soil samples were weighed out into 2 gram aliquots. The aliquots were suspended in 15 ml of sterile water, shaken into suspension and a 1 ml water suspension sample removed. The 1 ml aliquot was pipetted and a dilution series made twofold. Enriched agar media was poured into petri dishes and counted after three days incubation at ambient temperature.

Samples of 1.0 ml aliquots were treated with a 0.1 ml aliquot of Example 2 diluted 1:10 to test microbial kill. In various time increments, 0.5 ml aliquots were pipetted into petri dishes and 12 ml of enriched nutrient agar was added. The sample was allowed to solidify and measured after three days for colony forming units cfu's. The results are reported in the following Table 4.

TABLE 4

| Time (min.) | Desert | Mountain | Beach |
|---|---|---|---|
| 0 | 850 | 1,100 | 6,778 |
| 1 | 76 | 52 | 26 |
| 2 | 0 | 0 | 0 |
| 5 | 0 | 0 | 0 |
| 10 | 0 | 0 | 0 |
| 15 | 0 | 0 | 0 |

The results reported in Table 4 show that the composition of Example 2 killed all microbes isolated from soil samples obtained from desert, mountain and beach soils or sands. The complete kills were obtained within two minutes, where as, about 90% kill or better, was obtained. In the first minute of contact with Example 2.

The effectiveness of the compositions of the present invention in connection with the regulation of bacterial biofilms was evaluated in connection with Staph aureus, Pseudomonas aeruginosa, MRSA, mixed oral bacteria, Enterococci faecalis and E. coli. In each instance, a mature and healthy biofilm was cultivated on a gel surface to provide a matrix size of about a square inch or more. The starting biofilm was three days old and grew as an amorphous smooth surface gel-like mass owing to the mucous secretion of the adherent mass of bacteria.

Each biofilm sample was contacted with the composition of Example 2 at room temperature and at a rate of 10 ml per sample for three and 15 minutes treatments. After the treatment times, the biofilms were washed with phosphate buffered saline and fixed with gluteraldehyde. They were then prepared for scanning electron microscope (SEM) without otherwise affecting the nature of the test.

The following observations characterize the effectiveness of the composition to control and destroy the biofilm mass with kill of the bacteria species.

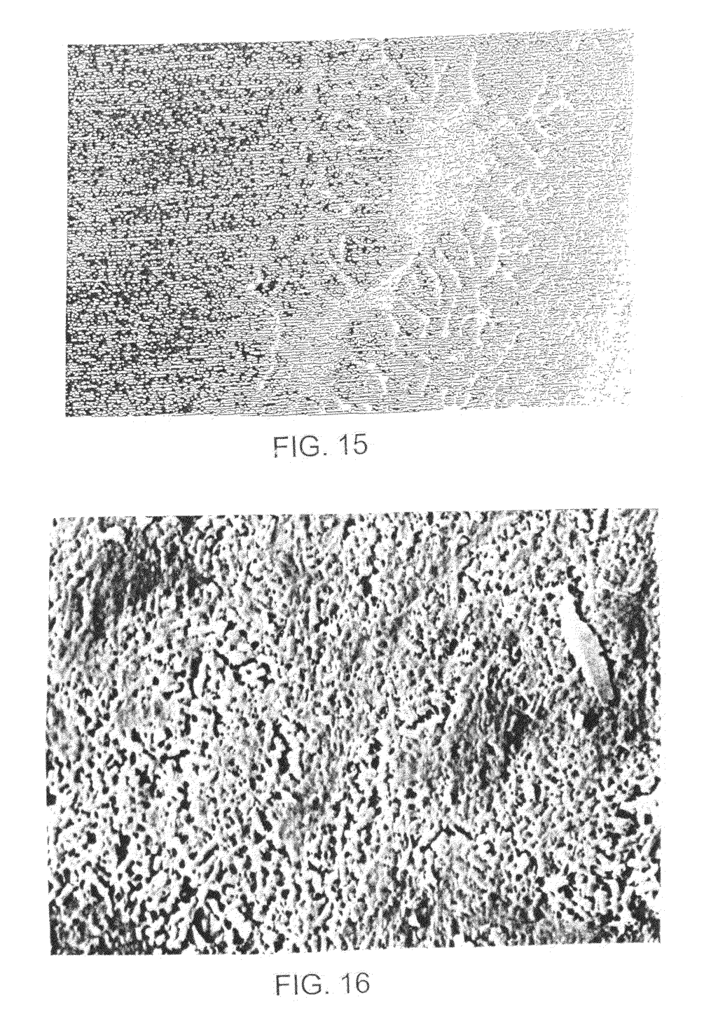

FIG. 15 shows the Staph aureus biofilm after three minutes treatment with Example 2 as seen at 100× magnification. In this instance, the composition was effective to dissolve the biofilm for the most part, and the bacteria were reduced to a planktonic state after 15 minutes, but not killed.

FIG. 16 shows the Pseudomonas biofilm after three minutes treatment with Example 2 as seen at 2000× magnification.

FIG. 17 shows the Pseudomonas biofilm after 15 minutes treatment with Example 2 as seen at 2000× magnification. Considerable damage and substantially complete kill has occurred to the biofilm.

FIG. 18 shows the MRSA biofilm after 15 minutes treatment with Example 2 as seen at 5000× magnification. A complete destruction of the bacteria in the biofilm is shown. The matter in the photomicrograph is the leftover slime that once covered the biofilm colony.

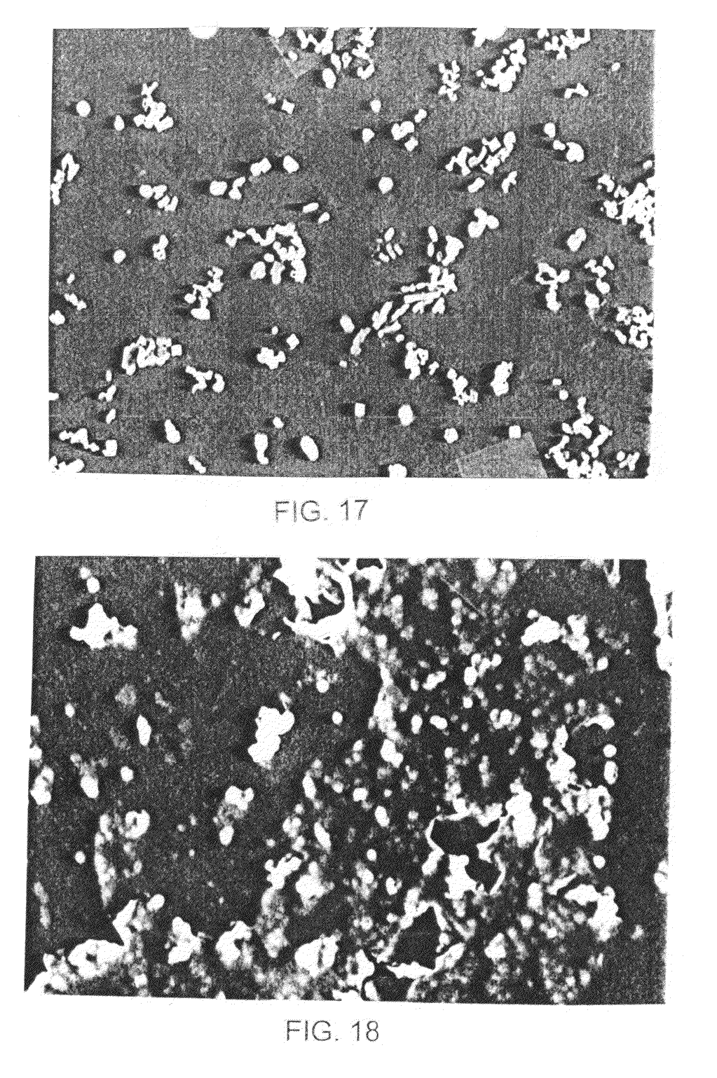

FIG. 19 shows the mixed oral biofilm after 3 minutes treatment with Example 2 as seen at 1000× magnification. As shown, the biofilm colony has been broken with parts reduced to a planktonic form. About one-half the biofilm was reduced to the planktonic state with very little bacterial kill.

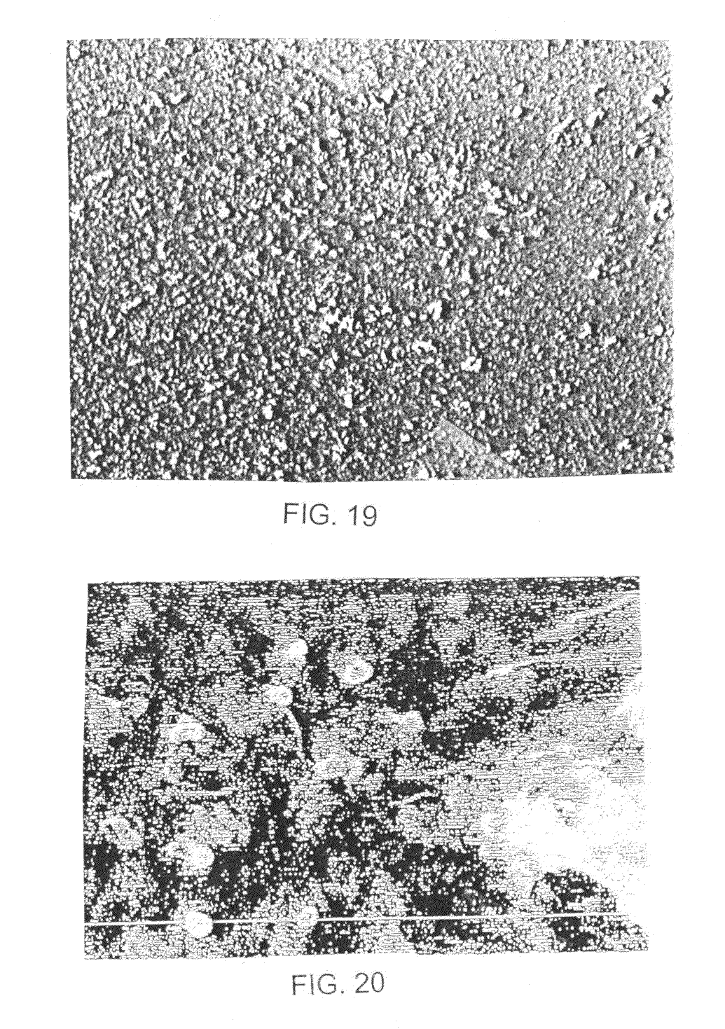

FIG. 20 shows the mixed oral biofilm after 15 minutes treatment with Example 2 as seen at 5000× magnification. A large part of the colony has been unaffected. About one-half the biofilm was reduced to the planktonic state with very little bacterial kill.

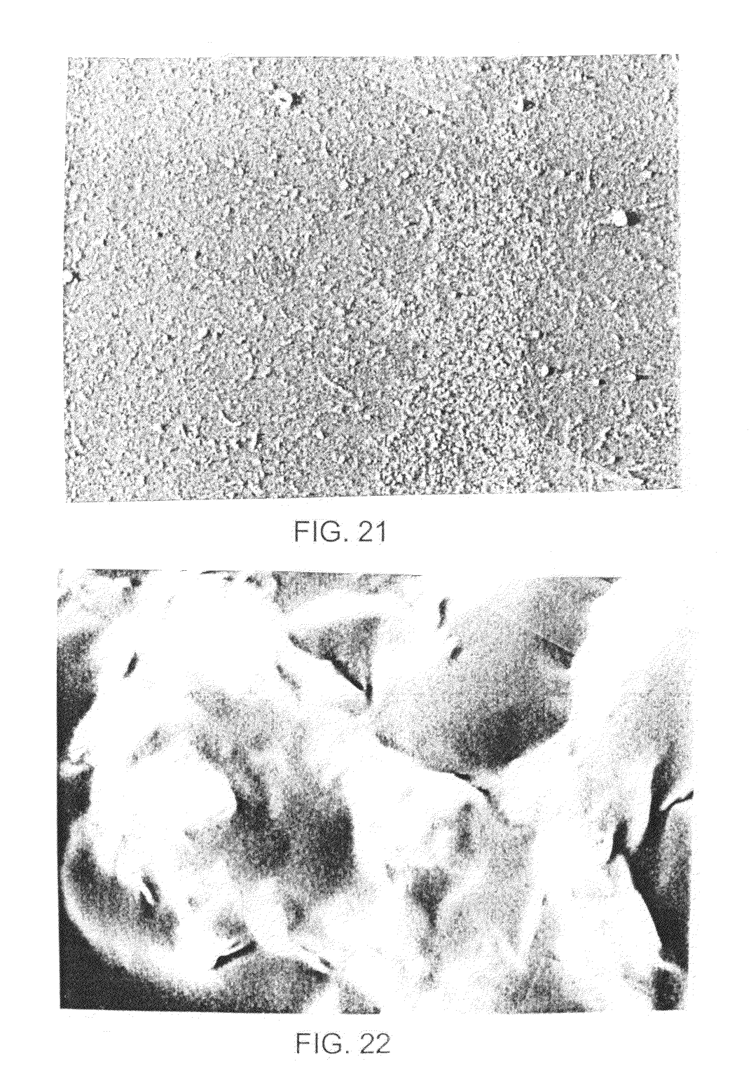

FIG. 21 shows the mixed oral biofilm of FIG. 21, but at 100× magnification to give a broader view.

FIG. 22 shows the Enterococci biofilm after 3 minutes treatment with Example 2 as seen at 5000× magnification. About one-half of the biofilm was destroyed. The remains of the biofilm slime are shown devoid of any bacteria.

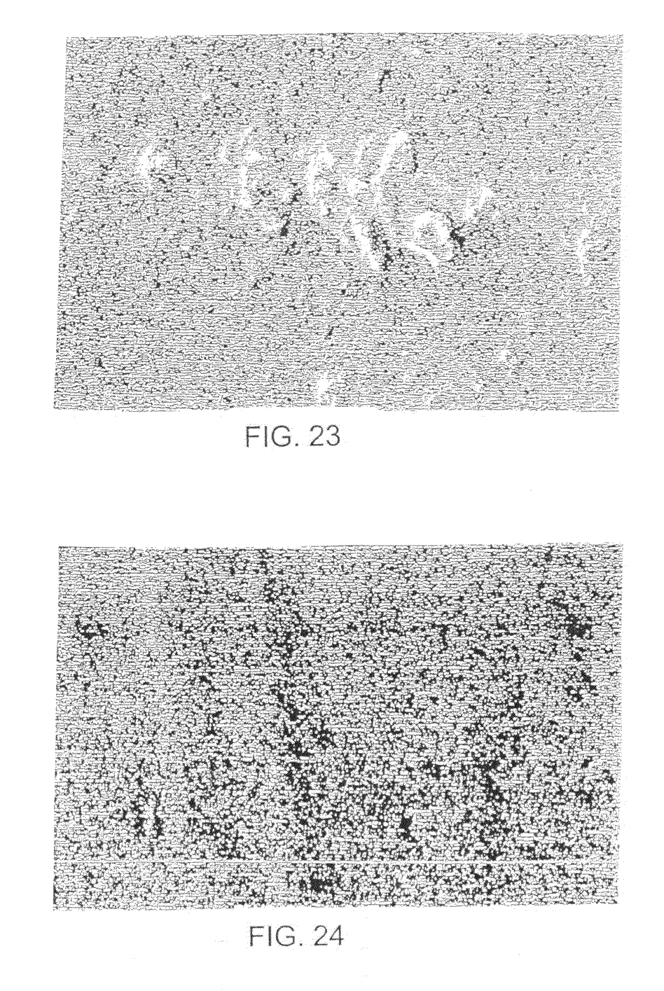

FIG. 23 is similar to FIG. 23, but shows another part of the remains of the biofilm as seen at 1,100× magnification.

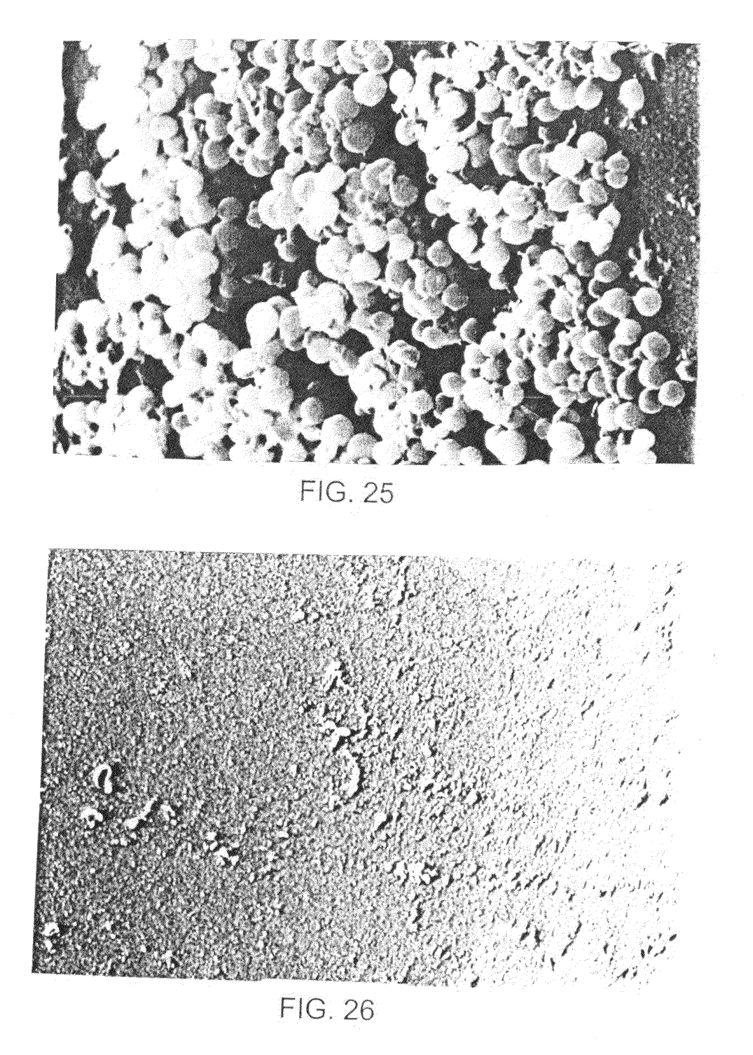

FIG. 24 shows the complete destruction of the Enterococci biofilm after 15 minutes treatment with Example 2 as seen at 100× magnification.

FIG. 25 shows the E. coli biofilm after is minutes treatment with Example 2 as seen at 5000× magnification. A noticeable breakup of the biofilm colony is noticed in three minutes and after is minutes the E. coli colony has been taken out of its biofilm state.

FIG. 26 shows the E. coli biofilm after is minutes treatment with Example 2 as seen at 2,000× magnification.

The compositions of the present invention are also useful for personal hygiene, as for example, a liquid soap composition. In liquid form, the composition may be dispensed using a conventional pump arrangement and a plastic container. To that end, the composition of Example 2 was evaluated as a soap and a shampoo to demonstrate successful reduction In microbe count in key body areas, such as the head, face, legs, arms and feet.

Test individuals included four males ranging from 17 to 66 years of age. One female was tested for hand cleaning.

The inventive compositions were compared with the following commercial products.
1) DOVE brand white bar soap by Unilever of Turbil, Conn., USA, and
2) Liquid antibacterial soap sold under the DIAL trademark by the Dial Corporation of Arizona, USA.
3) KIRKLAND brand shampoo marketed by Costco Corporation of Seattle, Wash., USA. This shampoo contains sodium lauryl sulfate, cocamidopropyl betain, aloe vera, jojoba oil, methylparaban EDTA, methylchloroisothiaolilnone and algal extract.

There is considerable variability in individual washing procedures. This includes both body wash and shampoo applications. In spite of such variation, the compositions were found to reduce microbial levels from every test site. The sites of highest microbe loads were hairy areas such as the chest, under arms and groin.

Baselines were established by swabbing at the end of the workday or in the morning. Swabs were inoculated directly on blood agar plates, or in the case of high counts, swabs were broken off in test tubes with 5 ml sterile saline, and mixed in a vortex mixture for 2 minutes to release bacteria from the swabs. Aliquots were then measured by dilutions and 0.5 ml was added to a blood agar plate. The mixture was spread by a plastic plate spreader and incubated for 48 hours prior to plate counts and cfu determinations.

TABLE 5
Head and Upper Body (cfu count)

| | | After Soap/ | |
|---|---|---|---|
| | Start Baseline | Shampoo | After Example 2 |
| Cheek | 489 | 312 | 67 |
| Nose | 3,027 | 2,905 | 190 |
| Chin | 110 | 80 | 56 |
| Hair | 212 | — | 57 |
| Scalp | 2,100 | — | 222 |
| Forearm | 57 | 32 | 11 |
| Bicep | 25 | 20 | 10 |
| Underarm | TNTC | 4,050 | 3,450 |

Using the foregoing procedures, additional evaluations were made as reported below in Table 6.

TABLE 6
Hair and Body Parts (cfu count)

| | | After Soap/ | |
|---|---|---|---|
| | Start Baseline | Shampoo | After Example 2 |
| Chest | 1,125 | 385 | 101 |
| Back | 127 | 96 | 55 |
| Buttocks | 17 | 12 | 6 |
| Thigh | 121 | 26 | 11 |
| Calf | 47 | 29 | 12 |
| Foot | 98 | 80 | 48 |
| Between Toes | TNTC | TNTC | 3,080 |
| Back of Hand | 47 | — | 1 |
| Palm | 212 | — | 2 |
| Hand Knuckle | 10 | — | 1 |
| Hand Nail | 1,600 | — | 960 |
| Thumb | 6 | — | 1 |
| Forefinger | 12 | — | 3 |

The compositions of the present invention are useful in connection with instrument sterilization in the field. Instruments tested included scissors, forceps, tweezers, dental burs, probes, explorers and clamps. Serrated edges, hinged devices and knurled ends were particularly examined to confirm whether sequestered areas could be disinfected.

The instruments were placed in trays containing 10⁸ bacteria per milliliter and allowed to remain in contact for 45 minutes. The instruments were then removed, air-dried, and placed in sterile tubes with various dilutions of Example 2 including 1:5, 1:10, 1:20 and 1:40. After incubating with Example 2 for various times, the instruments were removed, dipped in saline, and placed aseptically in sterile tubes of appropriate sizes containing sterile media and incubated at 35° C. for up to 8 days.

Tubes and positive controls could be visually detected by turbidity. Media containing purple base could be detected by observing a purple to yellow color shift via pH change by acid production indicating microbial growth. Growth was surveyed at room temperature and at 35° C. incubator temperature under aerobic conditions.

Positive control tubes showed turbidity at 24 hours and extensive turbidity at 48 hours. Under proper conditions, no growth was observed at eight days. In some conditions of lower-level kill at eight days, very few microbes per milliliter were detected, the worst case scenario being less than 10 microbes were found. Under the sterilization conditions, no turbidity or pH change is detected, nor any cfu's noted when 1 ml of test media was inoculated and spread on the surface of blood agar plates.

In the following Tables 7, 8 and 9, the reduction Strep pyogenes at day 8 after exposure to Example 2 for various times is reported.

TABLE 7
Reduction of Strep Pyogenes
Day 8 of Test After 5 Minute Exposure to Example 2

| | Turbidity | pH Shift | cfu's |
|---|---|---|---|
| −Control | None | None | 0 |
| +Control | Heavy | Yes | TNTC |
| 1:5 Dilution | 0/3 | Slight | 63 |
| 1:10 Dilution | 2/3 | Moderate | 3,050 |
| 1:20 Dilution | 3/3 | Heavy | TNTC |

TABLE 8
Reduction of Strep Pyogenes
Day 8 of Test After 5 Minute Exposure to Example 2

| | Turbidity | pH Shift | cfu's |
|---|---|---|---|
| −Control | None | None | 0 |
| +Control | Yes | Yes | TNTC |
| 1:5 Dilution | None | None | 0 |
| 1:10 Dilution | Slight | Yes | 693 |
| 1:20 Dilution | Yes | Yes | 7815 |

TABLE 9
Reduction of Strep Pyogenes
Day 8 of Test After 5 Minute Exposure to Example 2

| | Turbidity | pH Shift | cfu's |
|---|---|---|---|
| −Control | None | None | 0 |
| +Control | Yes | Yes | TNTC |
| 1:5 Dilution | 0 | 0 | 0 |
| 1:10 Dilution | ± | Slight | 16 |
| 1:20 Dilution | + | + | 1720 |

The foregoing data confirm that the composition of Example 2 is capable of disinfecting as long as sufficient time elapses for contact with the contaminated instrument. Presently, it appears that a minimum of about 15 minutes is required for complete disinfection to occur. For convenience, a device impregnated with Example 2 may be contacted with the instrument to maintain constant contact during procedure. A moist liquid bandage of the composition provides optimum results. For example, the instrument may be wrapped with a foraminous or fibrous carrier material impregnated with the composition and having an impermeable outer sealing layer.

It should be appreciated that the compositions themselves may be formed into integral bandages in situ. The compositions may be applied as a thin liquid film or as a foam and allowed to dry to a continuous thin film. For example, diluted compositions of Example 2 at concentrations ranging from about 2% to about 5% active ingredients will form a foam upon dispensing with mild agitation as resulting from hand the liquid from a container. Satisfactory results have been obtained with bottles marketed by Ainspray International Incorporated.

A measured pump volume of about 0.3 ml will typically treat a two to three inch long skin wound with a foamed layer of the composition resulting from direct pump-bottle application. The foam is temporarily sustainable at room temperature and atmospheric pressure. In a few minutes, the foamed composition spreads out and collapses to form a substantially continuous film or thin strip about 1 by 3 inches long. The thickness of the thin strip is estimated to be a few thousands of an inch.

A single bandage formed in this manner will last for one to two days, but the bandage may be applied two or more times daily. In two days, a typical cut wound is scabbed over. Initial tests indicate that the bandage is effective to prevent infection of wounds such as burns, glass or metal cuts or on a skin biopsy for a mole removal. It appears that rapid healing is promoted.

The compositions of the present invention are useful as antiseptics or disinfectants for treating of medical facilities per se. For example, over thirty medical, dental and laboratory facilities including operatories, laboratory equipment and waiting rooms were cleaned using the compositions in the form of foams, sprays and liquid as applied with a wipe cloth. The composition of Example 2 has shown excellent antimicrobial/cleaning powers at least equaling, but usually exceeding, other standard disinfectants.

The areas of highest contamination in dentistry were sinks, floors, high power evacuation lines and counter tops. Aerosol studies indicate that the higher the microbial count in water lines, the higher the surface count. Aerosol fallout is the source of surface contamination. Patients with high oral microbial counts also add greatly to the aerosol bioload during operative procedures.

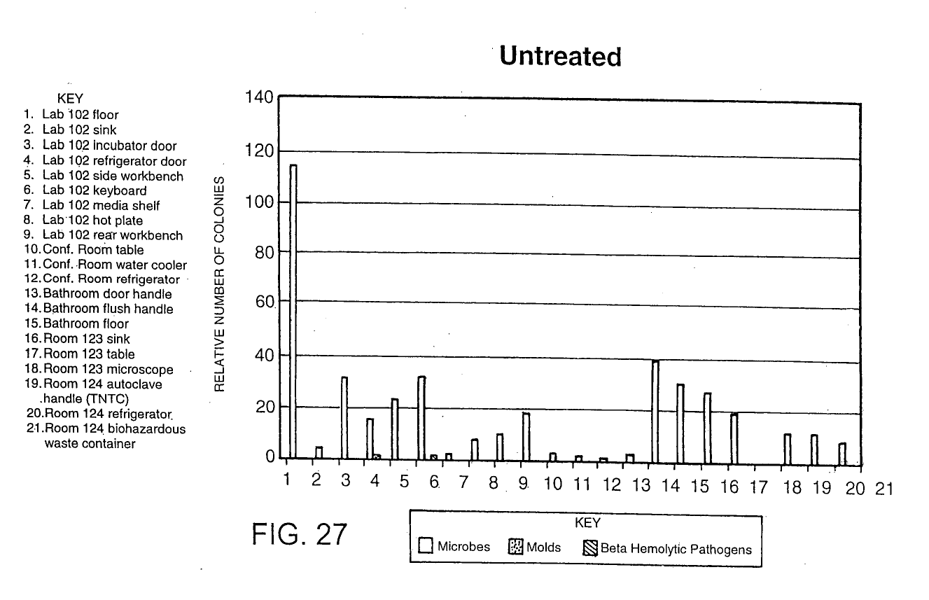

Referring to FIG. 27, a survey count of the microbes; molds and Beta hemolytic pathogens present in the indicated untreated areas of a tested dental facility is shown. The microbe count on the autoclave handle exceeded the report range, and next highest count of microbes occurred on the lab floor.

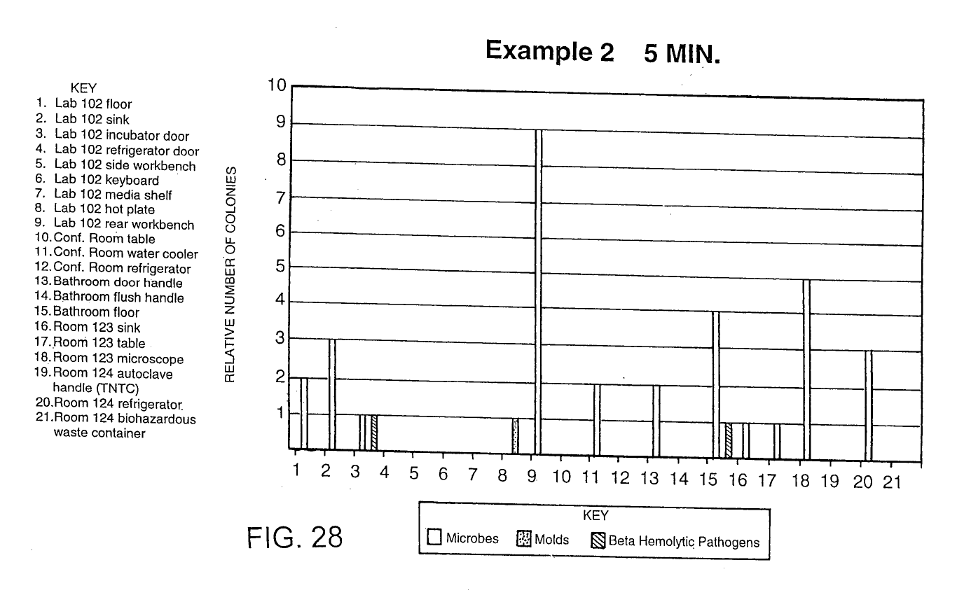

Referring to FIG. 28, a similar survey report of microbe count after five minutes following treatment with the composition of Example 2 is shown.

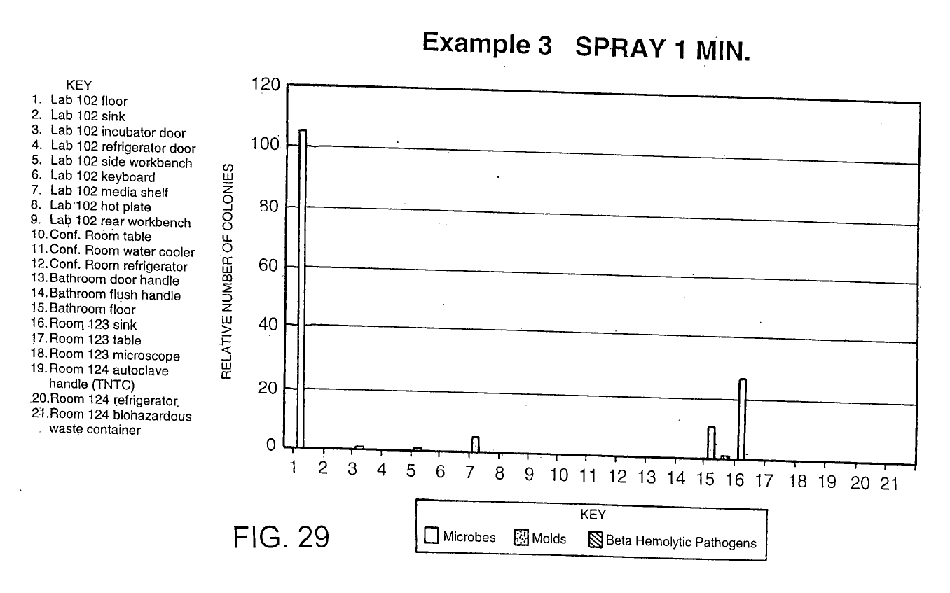

Referring to FIG. 29, a count of the dental facility is shown one minute after a spray application of a 1:10 dilution of the composition of Example 2. As indicated, a significant reduction in the microbe count occurs in all areas except for the lab floor.

The comparative use of bleach and the composition of Example 2 to clean the operatory lab and laboratory facilities with respect microbes, molds and pathogens is summarized in the following Table 7. As shown, Example 2 is as effective as bleach in reducing to substantially zero the microbe, mold and pathogen counts.

TABLE 7
MICROORGANISM COUNT

| | Untreated | Example 2 | Bleach |
|---|---|---|---|
| Operatory Lab | | | |
| Microbes | 5000 | 0 | 0 |
| Molds | 0 | 0 | 0 |
| Pathogens | 0 | 0 | 0 |
| Operatory | | | |
| Microbes | 5000 | 0 | 0 |
| Molds | 0 | 0 | 0 |
| Pathogens | 0 | 0 | 0 |

The compositions of the invention are also useful in connection with the operation and maintenance of dental hand pieces. The compositions may be added to the circulating water system for the dental hand piece to provide sterilization-disinfectant, antiseptic and lubricant properties during operation. Further, the severe conditions of the autoclave procedures heretofore used to sterilize dental hand pieces may be replaced by room temperature contact sterilization treatments with the inventive compositions. The foregoing use of the compositions significantly reduces expected maintenance repairs of the hand pieces.

The compositions may be added to a closed water circulation system for the dental hand piece to provide a fluid mixture having a concentration of active ingredients equal to about 0.1%. The fluid is circulated to the hand piece which impinges a stream of fluid onto the tooth surface being cut. Without detriment to the cooling effect of the fluid, the impinged fluid is dispersed and forms an antiseptic aerosol in the oral cavity with activities exemplified by the mixed oral flora tests reported in connection with FIG. 9. The sterilization effectiveness of the fluid is confirmed by cleaner evacuation traps for the water system believed to result from the inhibition of biofilm formation and reduced levels of microorganisms. The traps previously contained a gel-like biofilm, but the described use of the compositions results in a white powder in the traps that is believed to be the residue of the destroyed biofilms.

The compositions may be used at a concentration of active ingredients of about 1.0% to wash and soak the hand pieces in a room temperature sterilization process that replaces the previously used high temperature autoclave cycles. The hand piece is initially taken apart; sprayed with the composition to remove bulk debris and then allowed to set for 10 minutes. Thereafter, the sterilization is completed by soaking the hand piece in the composition for approximately 20 hours at room temperature. This sterilization process is believed to extend the lives of the elastomeric gaskets and fiber optic tube components as compared with autoclave treated hand pieces.

The compositions have a lubricous quality that provides effective lubrication of the rotating components such as the turbine and its rotational mounting assembly in the hand piece. In a long term test including multiple low and high speed hand pieces, the incident of expected replacement of the turbine and chuck assembly was reduced by 80%. That is, the seven hand pieces tested required replacement of six turbine and chuck assemblies during the test period. In comparison, it would have been expected to replace about 35 turbine and chuck assemblies in seven such hand pieces when used for a like duty cycle and time period with a water coolant and autoclaving in accordance with prior art procedures.

The compositions of the present invention are not toxic and do not result in cell damage at useful pH values in the range of from about 4 to 7.5 and suitably dilute concentrations.

The in vitro cytotoxicity of the composition of Example 2 was evaluated using cell culture system of C3H/10T1/2 C1 8(10T1/2) mouse embryo fibroblasts. The cells are grown in humidified incubators at 37° C. in an atmosphere of 5% carbon dioxide/air (v/v). 10T1/2 cells are thought to be a spontaneously immortalized, primitive mesenchymal cell line.

The cytotoxicity assays were conducted using standard methods in which 200 cells/60 mm dish were plated and five dishes were prepared for each concentration of Example 2 to be tested. In preliminary screening, it was found that a 1:20 dilution of Example 2 reduced the plating of the cells to 77.8±5.3%. At a 1:2 dilution, the plating efficiency of the cells was reduced to 0% with all cells being killed.

The cytotoxicity was determined to be dose-dependent. It was determined that dilutions in the range of 1:10,000, 1:2,000, 1:1,000 and 1:200 caused little or no cytotoxicity. The plating efficiency of 10T1/2 cells for the following dilutions were determined.

| Dilution | Plating Efficiency % |
|---|---|
| 1:100 | 94.0 |
| 1:50 | 85.4 |
| 1:33.3 | 83.3 |
| 1:25 | 74.0 |
| 1:20 | 67.7 |
| 1:10 | 16.1 |
| 3:20 | 0.0 |

The cytotoxicity of the composition of Example 2 is therefore dose-dependent in this concentration range.

The LC50 value which reduces the plating efficiency to 50% of that of control cells is estimated to be between a 1:25 and 1:10 dilution of a solution containing 50.0 ug/ml of active ingredients which corresponds with a concentration between 10.0 ug/ml and 25.0 ug/ml. Similarly determined LC50 values for acetaminophen, aspirin and borax are 1,000 ug/ml, 1,500 ug/ml and 2,000 ug/ml.

### Formula and 5 Studies

### Animal Studies

### Phase 1

### Eyes

Higher concentrations of the test solution showed extreme irritability in rabbit eyes. The animals were examined for an additional two weeks on a daily basis and the irritated eye sequalae disappeared after applications were discontinued. High concentrations, 1:4 to 1:16, took eight to ten days to clear up; but concentrations of 1:32 cleared up in five days. Examination of animals showed no scarring or opacity of any external eye structure. The animals were sacrificed at the end of the study.

Dilutions of 1:60 were dropped in one eye and controls of sterile phosphate buffered saline (PBS) were added to the other to test whether more dilute concentrations would demonstrate that less irritation would occur. T 1:60 concentration still demonstrates antimicrobial effects. That higher dilution (1:60) was tested for irritability.

The first 10 days, the animal was dosed with 2 ml pet ocular test in the a.m. Reddening appeared at the rim or red part of the perimeter of the eye socket only. This minor irritability disappeared daily in 30 minutes-1 hour, even the control showed slight signs of irritation. After 10 days testing the dose was increased to twice a day (a.m. and p.m.) and the same short term irritation in the red rim of the eye only appears and then disappears. This study has continued and will go on to the end of the month. This demonstrates a potential for eye use at diluted concentrations. In the future, we will test 1:80-1:100 for effectiveness against key microbes.

### Vaginal Effects

Two animals were continued to be subjected to 2× daily vaginal swabs of 1:8 solutions. At the end of the six weeks, one rabbit was sacrificed and autopsied for vaginal ulcerations, or other abnormal tissue disturbances as a possible result from treatment. The vaginal mucosa visually appeared to be perfectly normal.

A large sample of vaginal tissue and muscle was fixed in 38% formalin for later histological exam. An additional rabbit was continued in the daily swabbing for a longer term study, most likely to be terminated.

### Oral Mouth Rinse Study

One animal was sacrificed and autopsied six weeks into the study. The second rabbit is continuing twice daily mouth rinses of 5-10 mls. The sacrificed animal's oral tissue, gingiva, tongue, mucosa and palate all were normal in appearance. Samples of tongue, palate and gingiva were fixed in formalin. The gullet was examined and found to be normal and samples formalin fixed. The stomach, duodenum, and small intestine all appeared normal and tissue sample fixed and saved. The liver, pancreas and kidneys and rectum also appeared as health tissue. There was complete absence of lesions, spots, tumors, cysts or other abnormal signs.

The mouthwash study shows a substantial oral potential. If one considered apthous ulcers or halitosis, primarily a tongue microbe problem, there are a lot of possibilities.

Because of the value of long-term oral exposure, testing will continue on one rabbit on a long-term study to help still arguments regarding long-term mouth rinse use.

### Wound Healing Study

Two prime rabbits were selected for this study. Two-inch incisions were made on either side of a rabbit. Rabbits were clipped and shaved to expose skin and rubbed with alcohol prior to excision. One cut side remained untreated except for sterile PBS, pH 7.2 application is used as a control. The other test side is treated with a 1:8 dilution of the test solution.

The excisions were:

1. Between epidermis and dermis—Termed mild injury

2. Through the dermis to flesh—Termed major injury

Applications of PBS and 1:8 test solutions were made twice daily. Photographs were done periodically. This test is to be continued to better to determine results.

### Animal Injection Study

After three weeks of injections, one half ml of a 1:8 solution in the marginal ear vein, or in multiple areas in the back, every other day, the injections were discontinued and the animals monitored. In three days, any sign of injection in the ear vein was gone and the ear took on an untouched appearance. Small knots in the back adsorbed out in about 10 days and no sign of them exists now.

### Microbiology Study

Studies were initiated on the killing potential of spore forming organisms. These studies were tried for several reasons:
Anthrax of animals is a spore former.
Tetanus of humans and animals is a spore former.
Spores are the most difficult things to kill in the known microbial world.
All sterilization tests are based on killing spore formers.
Bacillus sterothermophilus (Z line=30 minutes) is the toughest to kill. This one takes 30 minutes of autoclaving to kill.

Microbiology results show that dilutions throughout 1:32 kill spores from B. subtilis, and B. sterothermophilus, epidermis and dermis with little blood seepage. The wounds front (less tension and stress) and rear (more tension and stress) were on both sides of the animals. The left side was treated with a control two times daily, phosphate buffered sterile saline. The right side was treated with 1:8 dilution of Solution A. After two weeks, healing occurred with no detectable difference between treated and untreated animals.

### To Further Clarify Wound Healing

A second set of rabbits were prepped with a five inch wound made on a single side of each (no leg stretch) One wound was shallow while the other wound was a deep gash through the hide of the animal to flesh. Much blood flow was noted. This was cleaned and the front two inch portion treated with PB-saline, the middle one inch section left alone as untreated control, and the last two inches treated with 1:8 Solution A. Sterile gauze wound patches were placed over the lesions for a few hours until bleeding ceased. The animals were treated with PB SD, nothing and Solution A twice daily. Results were photographed and recorded. Solution was added to drain over the wounds by Pasteur pipette, 2 ml each. As of day five, no differences in healing have been noted in shallow or deep wounds. Deep wounds are scabbing slightly at the edges. The wounds are extensive and the healing process greatly challenged. The shallow wounds are scabbing over, but again with no apparent increase in health by the Solution. This study is ongoing.

Germicidal Studies Continued. Spore Killing Effects of Solution A. Raven Prospore Biological Indicator Sports (Lot 5) were utilized in a concentrations ranging from 10⁴ to 10⁵ to 10⁶. Two types of Bacillus Stearothermophilus spores were used. This bacteria ATCC #7953, is the standard for steam or autoclave sterilization testing at 121° C. Two different strains were used, one designed for 15 minutes kill (Z=15) and the second heat resistant for 30 minutes (Z=30).

Cells were tested in blood agar or pour plates made from standard plate count methodology using a special media consisting of nutrient broth, supplement with 1 gram each of tryptane yeast extract, brain-heart infusion and trypticase soy broth. A gelling agar of 0.8% was added too.

### Summation of Initial Animal Studies

Mouth Lavage: Two rabbits continued on daily mouth lavages (1-5 ml). One animal is quite tolerant of swallowing the liquid. These animals are 2½ A months into treatment with no adverse effects noted in eating, excess water usage or excrement. Anesthesia allows for extensive oral examination and tongue, gingiva, soft and hard palate all appear normal.

Injections: One rabbit still undergoes injections intramuscularly twice a week. Knobs of irritated tissue appear which might be called small cysts. These disappear within a week after cessation of shots. One animal was sacrificed and no abnormal organ lesions were found in the GI tract. The second animal (two months in study) will be sacrificed this week. The best conclusion is, virtually nothing permanent going on. Microscopic exam will show more on tissue sections in the histology/pathology lab.

Eye Study: One rabbit has been undergoing eyewashes with 1:60 dilutions of solutions A, 1-2 ml drops, daily. This animal has been studied this way for seven weeks now. Slight redness occurs for 10 minutes or so then disappears.

In one instance, the animal was anesthetized and an eye was flooded with 1:60 Solution A and left wet for five minutes, until the animal recovered. This huge volume long term flooding still only induced minor reddening for 10 minutes. These results indicate that large volume, long time exposure has roughly the same irritability as a short term minimal eye wash. This study is still ongoing to look for long term exposure effects. An additional month observation showed no negative effects.

Wound Healing: Two rabbits were prepared for wound healing tests by scissors, electric clipper and finally shaving with soap and water while under anesthesia. Two inch wounds were made, on the hindquarter where stretching occurs and one on the upper back of each animal. The incisions were shallow separating the solidify the plates. Prior to pouring, the sterilized liquid media was kept at 46° C. in a water bath.

Aliquots of microbes containing known amounts of spores or late log phase, early stationary phase B. sterothermophilus cells containing spores (ca. 25% of the vegetative cell population) were used in spore killing experiments.

Spores from Raven Biologicals were titrated again in this lab to verify their data. Samples of spores or living cells were mixed with aliquots of 1:4 Solution A and assayed on a time course study. The 1 ml spores; 1 ml Solution A yielded a 1:8 Solution A final concentration. At various time points, e.g. 5, 10, 15, 20, 25, 30 minutes, the test materials were diluted in sterile water to 50 ml yielding a 1:400 dilution of the chemical. A 1 ml aliquot of this diluent sufficient to stoop kill power of the solution A was added to 15 ml liquid agar media (another 10 fold solution to eliminate chemical kill while under incubation). Results were compared with control dilution in water instead of chemical for a standard control.

Results of drop tests in plates containing 100,000 Bacillus on a bacterial lawn showed the antimicrobial activity down to 1:40 dilution of Solution A. Titrations of Z=30 Bacillus revealed 5×10⁶ spores/ml Z-15 cells contained 3×10⁶ bacteria/ml Z=30 spores exposed to Solution A revealed the following pattern of kill.

| Time Exposure | Number of Bacteria |
|---|---|
| 5 min. | 5.0 Million |
| 10 min. | 4.5 Million |
| 15 min. | 4.0 Million |
| 20 min. | 3.5 Million |
| 30 min. | 100,000 |
| 60 min. | None detected |

Similar results were found for Z=15 spores.

The cultured Bacillus (not pure spores) showed reduction to 350,000 in 15 minutes. These results are being repeated, but what has occurred suggests extensive killing power of a 1:12 Solution A vs. spores.

### Animal Studies

### Phase 2

### Direct Ingestion:

One rabbit has been put on Solution A 1:40 via a water dish ad libidum. No other fluids are provided. This is being done to ensure a massive injected dose of Fluid A.

Results: After 3 days, the rabbit appeared to have refused further fluid. We waited an additional two days and the rabbit ceased drinking. If after today the rabbit refuses to drink, we will provide him with water for 2 days, then go back to a 1:40 solution. The rabbit's behavior has been normal and feces appear normal. No interruption in feeding or physical activity is noted, merely refusal to drink.

An additional rabbit is being put on 1:60 dilution as well as the current 1:40 study. We are testing to see if the more dilute solution is more acceptable to the animal.

### Injection Study

The rabbit was injected in multiple intradermal sites on the back, and injected IV as well. The animal has been sacrificed and the autopsy has revealed no visible organ damage nor bowel damage. Biopsies have been taken and fixed in buffered Formalin for histologic examination.

### Wound Healing

The two rabbits, one with a deep wound and one with a shallow wound shows no difference in healing time. The comparison was made between Solution A treated, untreated and phosphate buffered saline control. We found no initial increase in health rate during the first 3 days, the middle range period, the next 7 days, or the final stage (the final 10 days) of swabbing and hair regeneration.

### Force Feeding of 1:40 Solution A

One rabbit was held daily and 3-5 mls of solution was force pipetted down the animals throat. This animal was allowed to drink water and Libidum so a combination of Solution A and water was used. Again, an autopsy revealed no adverse reactions in organs or intestines. This animal was under testing for 3 months.

### Eye Study

After 2 months of 1:60 Solution A eye drops 2× per day, no eye damage is noted. This animal is being maintained presently for observation, with no treatment.

New rabbits are being purchased for a burn study. Two animals will be shaved and burned with a 4 inch burn while under anesthesia. One will be a control PBS only. The other will be treated with 1:40 Solution A. This study will view burns vs. wounds for healing or infection control. A third animal will be infected with Pseudomonas, a common hard to control microbe, often associated with burns and a burns control center's greatest fear. A fourth animal will be inoculated with Pseudomonas and treated with Solution A to see if prevention of infection is possible.

The possibility of Solution A in burn treatment or for room disinfection is opened by this study. Extraordinary precautions are taken in burn wards to avoid infection. Walls are painted with a silver solution as silver is antimicrobial. Pseudomonas infections in burn wards are deadly and greatly feared by burn ward physicians and nurses.

### Microbial Studies

A second spore former study was initiated with Bacillus subtilis, a body temperature spore former. The first round of results of kill show that solutions of up to 1:70 are lethal to this microbe. A formal, numerical kill curve has been started by B. subtilis. Pseudomonas has been obtained and are under cultivation to initial animal burn studies and time course kill curve studies.

The solution appears harmless if swallowed. It might be an excellent purge system for this and other applications. A dosage of 2 ml/use is likely proper.

Solution A was Applied to Small Animal Problems.
Initial study: One cat is under test
Condition: Ear sarcoma with an associated microbial infection. Nasty condition.
Treatment: 2× per day swabbing Solution A.
Animal Reaction: Discomfort-acted as if it burned on delicate, exposed tissue.
Results: The microbial infection resolved in 4 days. The sarcoma shrinks down about 25% whether an oncogonic effect or an antimicrobial effect is not known yet.

Large Animal Studies will be pursued. A vaginal douche for large animals is expected to be effective.

On wounds, this is key: The solution does not have to promote healing, rather it should avoid infection and not block healing. As it turns out, iodine solutions and nitro furosans inhibit cell growth in the key treatments. Something better is needed. This is of extreme importance for human use too.

The present invention appears to meet this criteria.

### Animal Studies

### Phase 3

Animals were injected subcutaneously in the back and IV. After two months of injections, O.25 ml, 3 times a week, the animals were autopsied and no observable abnormal tissue structures, lesions or spots were noted on the tongue, trachea, mouth, stomach, large or small intestine, liver, spleen or kidneys. The heart appeared to be slightly soft, not firm muscle. Samples of key organs were excised and placed in buffered Formalin fixing solutions for histologic examination.

An animal was fed a 1:60 dilution of Solution A as its sole liquid source (replacing water) for 30 days. In the first three days, the animal balked at the Solution A diet and reduced its fluid intake about 50%. After the three day period, the animal increased ingestion of Solution A test formula and after two weeks drank freely.

After 30 days, the animal was sacrificed and autopsied. The oral cavity, tongue, esophagus, stomach, liver, spleen, kidneys, pancreas, and intestines were visually observed and no abnormalities, including lesions, punctures, colon alterations were noted. Tissue samples biopsied were taken for histology.

When the heart and pericardial space were examined, this organ was spongy rather than firm. Tissue samples were taken for gluteraldehyde fixation and subsequent histology and pathology study.

An additional rabbit was started on the regimen for another 30 days to compare with the results of the first animal.

### Burn Study:

Infected with Pseudomonas aeruginosa, three rabbits were shaved on the back, cleaned and anesthetized. They received 5 inch burns between a first and second degree nature with hot wires; three experimental sets were performed.

Controls: One half each of the lengthy burn was treated with phosphate buffered saline, pH 72. The other one-half was treated with 1:16 Solution A. The animals were monitored and photographed daily to ascertain whether any rapid healing of burns occurred with Solution A.

A second animal was burned and inoculated with Pseudomonas aeruginosa, a common burn ward microbial problem. One-half of the wound was cleansed with 1:16 Solution A immediately and the other half wound untreated as the positive control—a prophylactic study.

A third animal was burned and inoculated with Pseudomonas, but this time the animal was untreated for 48 hours to establish the early infection. After 48 hours, the animals were then treated with Solution A 1:16 and PBS, leaving the second half of the burn as a control. This was a therapeutic study testing whether the animal could recover with the help of Solution A after an infection had already started.

### Results:

Controls: The treated burn control appeared to accelerate healing for the first 2-3 days, then all appeared equal in healing.

$$\frac{\text{?}}{Solution A Treated} / \frac{\text{?}}{PBS Treated} Burn line$$

$$\text{?} \text{indicates text missing or illegible when filed}$$

Prophylactic Study: No infection started after the first 10 days and the treated and untreated animals appeared the same.

Pseudomonas inoculated whole length.

Treated with Solution A/Untreated Control Burn line

Immediately after inoculation by Solution A.

Therapeutic Study No infection started after first 10 days and the treated and untreated animals appeared the same; healing went along at the same rate.

Pseudomonas inoculated whole length of burn.

Treated with Solution A/Untreated Control Burn Line 48 Hours after Inoculation

The animals were observed closely throughout the day and found to be capable of twisting around to be able to lick their burn wounds. The animals were far more agile and flexible than conceived.

Conclusion:
Tongue cleaning is very efficacious in treating burns and wounds.
The animals are very tough.
All of the above.

Another set of studies was initiated, but this rime the back of the neck was shaved, cleaned and treated. The neck position eliminates the animals' ability to cleanse the wound by licking, even though a rabbit's tongue is about 3 times longer than it appears.

In addition to repeating the previous three experimental models, a fourth was set up utilizing a mixture of subgingival microbes.

$$Burn \text{?} \frac{Subgingival microbes / No treatment}{Inoculated immediately} \begin{matrix}Mix of Microbes \\ Untreated for 24 hours\end{matrix}$$

$$\text{?} \text{indicates text missing or illegible when filed}$$

### Veterinary Study

An ongoing study has been pursued with white line disease; a softening of hooves. The causative agent has been isolated in the lab and will be sent out for identification. Occasionally a mold appears, an Aspergillus species, but it appears to be not part of the main equation.

In vitro tests on Solution A, 1:16, on the bactericidal capabilities against the hoof microbe has shown effectiveness in kill down to 1:80 dilutions. Based on the cidal capabilities, farriers (shoers) have been recruited to try this on horses and mules. One study has been going on for a month. A 1:8 dilution of Solution A is being squirted into the underhoof where the shoe is. The farriers change shoes about every two months and will not remove them until that time, so we must wait. Softening occurs under the hoof where the shoe is. As I see this developing, if we get some good results as indicated by the in vitro studies, and we get this into the hands of several competent farriers with a protocol which can be followed, you will have a niche market. The problem is acute in the industry and the market very large.

### Continuing In Vitro Studies

Pseudomonas kill rates were analyzed with the modified antibiotic dilution inhibition assay used previously. Five microliter aliquots of test dilution of Solution A were dropped on Pseudomonas swabbed blood agar plates and brain heart infusion agar plates to test the cytotoxic efficacy. Results showed that dilutions of 1:75 Solution A were capable of killing Pseudomonas effectively.

### Animal Studies

### Phase 4

### Veterinary Studies

White Line Disease (Soft Hoof Rot).

After 6-8 weeks of sporadic application of Solution A diluted 1:10, the show was removed for replacement. The disease was found to be completely eradicated. Placement of a new shoe showed no softening or damage. The new shoes were nailed into solid hoof structure with no problem. Trimming revealed no problems. Additional farriers are being recruited. The response of the farrier was one of being stunned by the result. The usual response was “what is in that stuff?” We have solid potential here. Discussions are underway for other modes of application, including Solution A impregnated wax to coat the hoof. Dose delivery systems with plastic individual dose unit containers could be used. For example, the average dose in about 5 mls.

Absorbent sponge material and even bucket soaks are alternative methods. One method is described below.
1^{st} 3 Days: Pour contents of this package into a 5 gallon bucket filled with water and soak infected hooves daily for 10 minutes.

### Next Week

7 Days: Application of wax impregnated material.

### Next 6

Weeks: Follow up with daily applications of ampules until next shoe change.

Cat Ringworm

A group of kittens that developed ringworm were treated with Griseofulvin, an antifungal cream—no success; antibiotic resistance. Iodine treatment had no effect. Solution A (1:16) was topically applied three times daily for three days. By the end of day two, the ringworm was arrested and in major regression. By day three, the disease was resolved.

This is a treatment, which works when other treatment have failed. Griseofulvin, and even iodine, virtually the ultimate chemical killers were ineffectual, yet Solution A was safe and effective.

### Rabbit Studies

### Burn Studies: Continued

Animals were shaved (prepped) on the backside of the neck where they were unable to lick their burn wounds. Red-hot ⅛″ thick wire rods were used to burn animals just to the beginnings of red tissue. Animals were completely anesthetized during the process and Motrin tablets were ground and added to their drinking water supply to help alleviate post-treatment pain. The burns were four to five inches long. The experiments were run for three weeks.

The following studies were performed:

Control—no microbes inoculated

$$\frac{\text{?}}{1 \text{:} 16 Solution A} / \frac{\text{?}}{Phosphate buffered saline ( PBS )} Burn line$$

$$\text{?} \text{indicates text missing or illegible when filed}$$

Solution A was swabbed onto the left half of the burn daily. PBS was swabbed onto the right side of the burn daily.

Results

The moisturizing effect aided in healing and no external infection set in, in either control. At the end of three days, it was noted that the Solution A treated control had quickly scabbed over and that the healing process was accelerated. After one week, the healing process was basically over in the Solution A treated area, while the PBS treated area was progressing nicely. At the end of three weeks, both controls had sealed and were growing hair over the wound.

Summary

In the early healing stages without infection, the Solution A treated area had enhanced burn healing. This was probably due to stopping minor wound infection, and/or genuine promotion of wound healing.

Pseudomonas infected burns: Early treatment or prophylactic treatment. Pseudomonas (ca 3/10⁶ cfu's) were inoculated the entire length of the burn by swabbing. One half of the wound was immediately swabbed with solution A, the other half was left untreated after Pseudomonas inoculation.

$$\frac{\text{?}}{\begin{matrix}Immediately treated with \\ Solution A ; swabbing 1 \text{:} 16\end{matrix} } / \frac{\text{?}}{Untreated} \begin{matrix}Pseudomonas inoculated \\ burn wound\end{matrix}$$

$$\text{?} \text{indicates text missing or illegible when filed}$$

Results

The treated area never developed an infection and continued to heal the same as the Solution A control. These results indicate that after an initial high, Pseudomonas contamination of the wound, Solution A as a swab wash inhibited microbial growth and development of an infection. Healing the untreated control, on the other hand, revealed scab inhibition, pus and soft tissue infection. This proceeded for 12 days when scab formation started and the animals' own immune defense mechanisms took over and eventually resolved the infection.

Conclusion

When used immediately after an exposure to a burn wound, the product is efficacious. Early utilization enhances early cure.

### Therapeutic Utilization of Solution A on Psedudomonas Infected Burns

In this case, therapeutic means that the infected burn was allowed to remain untreated for 48 hours, then one half of the burn was treated.

$$\frac{\text{?}}{\begin{matrix}Solution A 1 \text{:} 16 \\ after 48 hours\end{matrix} } / \frac{\text{?}}{No therapy} \begin{matrix}Pseudomonas infected \\ burn for 48 hours prior to \\ Solution A treatment\end{matrix}$$

$$\text{?} \text{indicates text missing or illegible when filed}$$

Results

Pus formation and inflammation occurred within the 48 hour period when no treatment was started. Infection appeared throughout the entire length of the burn. After the second day, treatment started with Solution A by swabbing one-half of the burn. Within two days, the infection area under treatment started to resolve the inflammation and scabbing over was proceeding. The untreated area continued to fester. After two weeks, the treated area was well into complete healing, whereas the untreated area was still inflamed, although healing had started.

Conclusion

Even after a Pseudomonas infected burn has an established infection, Solution A is capable of stopping it allowing the healing process to start immediately. This observation is significant. No scarring or aberrations were noted. The wound smoothed over and fur covered the lesion.

Infection with Subgingival Flora

Bite wounds are often the most difficult to treat. Therefore, a subgingival culture of mixed oral flora was inoculated into an additional burn wound. The culture contained a ca 3×10⁸ cfu's. The burn was inoculated and allowed to infect for 48 hours prior to treatment with Solution A 1:16.

$$\frac{\text{?}}{Solution A treated} / \frac{\text{?}}{Untreated area} \begin{matrix}\begin{matrix}Burn infected with \\ subgingival flora\end{matrix} \\ for 48 hours\end{matrix}$$

$$\text{?} \text{indicates text missing or illegible when filed}$$

Results

The wound started oozing fluids and pus at the end of 24 hours and inflammation set in. Therapeutic treatment of one-half the wound site was started at the start of day three. Within 48 hours the lesion started healing the closure and scab formation started. The lesion remained infected in the untreated portion for an additional 7-10 days. Eventually, in two to three weeks, even the untreated areas regained resolution and healing.

### Conclusion

Solution A 1:16, is capable of stopping oral flora infection of mixed oral flora infections of burn wounds, rapidly and cleanly.

Solution A, therefore, has exceptional antimicrobial properties.

### Rabbit Studies

Two rabbits were placed in test in conjunction with the horticultural studies.

Animals were fed sprayed (1:16 Solution A) green grasses, weeds and rabbit pellets as food. This was done in order to see if ingested food sprayed for agricultural reasons would be detrimental to an animal's health. The study went on for 3½ weeks.

A rabbit was sprayed in the face daily with 1:16 Solution A to determine if an agricultural spray hitting an animal could cause deleterious effects to skin, eyes, fur or internal organs.

Results

The dry and green food sprayed with Solution A did not visibly inhibit the animals eating habits, increase her thirst or alter her daily habits. Upon sacrificing the animal, no evidence of intestinal tract injury was noted. Other organs such as heart, lung and kidney were also unaffected. The heart muscle, however, appeared softened and had an imploded look. This was reminiscent of the animal's heart that ingested Solution A and provides warning of potential ill effects by ingestion. Another rabbit is in the beginning of the third week of testing in drinking 1:60 Solution A to verify. Samples of lung, stomach and kidney were fixed in 37% Formalin for later pathology exam. Often times, a substance ingested chemically complexes with foodstuffs and metabolic parameters, then transforms the complex into pathologic of carcinogenic complexes. In this short term study, this was not noted.

A rabbit was sprayed in the face daily to ascertain whether aerosolization affected the eyes or lungs primarily, but the general respiratory system as well. No abnormal functions occurred during the one month study. No eye or mucous secreting system was adversely affected and no external lesions were noted including the eye. Autopsy revealed no organ aberrations with regard to tissue appearance. Lung tissue was saved for pathology and fixed in Formalin.

Conclusion

In these short term studies, there were no gross or detectable lesions, internally or externally with Solution A aerosolized into an animal's face or sprayed on its food, both dry and green, with the exception of heart tissue as mentioned above. This is no guarantee of long term effects or subtle change, but on the whole the animals were quite tolerant of the treatments.

### Microbial Kill Rates

Iodide vs. Solution A & 5

These tests were performed to determine, in a comparative way, the antimicrobial properties of Betadyne and both Solution A and Solution 5. Various concentrations of Betadyne and the Solution were added to constant concentrations of several bacteria and allowed to interact for several time periods, e.g. 0, 2, 5, 10, 20 and 30 minutes. At the end of each time period, an aliquot of the microbe-disinfection mixture was removed and plated out in a pour plate for colony number determination. In essence, data was obtained on the survival of microbes vs. time and concentrations of disinfectants.

Procedure

Concentrations of microbes ranging from one million to ten billion were aliquoted into 4.5 mls of media (high organic load). The media was composed of 1% yeast extract, 1% trypticase soy broth, 1% glucose and 4% nutrient broth. Agars were made to 0.8% for pour plates. Cultures were in late log phase of growth 18-24 hours when testing began. Various concentrations of disinfectants were added at a volume of 0.5 ml to 4.5 ml microbe-media mixture. At the various time points, 0.2 ml aliquots were removed and added to Petri plates. Media agar mixtures were added (15 ml) to each plate and then rotated to provide a uniform mixture for counting after 48 hours incubation at 36° C. or 58° C. for Bacillus stearothermophilus spores. Colony counts were then made and CFU's plotted. Most experiments were run in triplicate on several days.

The two key microbes reported here were Candida albicans, a gram+yeast and a white line hoof disease which is a large-gram rod. Controls of dilutions of disinfectant were tested to ensure the mixture put into nutrient agar did not cause a killing effect over a 24-48 hour incubation. Controls showed that the dilution of disinfectants in nutrient agar carried no residual effect into the test plate. In other words, once poured into the test plate, the action of the disinfectant stopped.

### Heat Enhanced Effects of Solution A & 5

There are many potential applications for Solutions A and 5 in industrial or instrumentation applications. One such application is with handpiece sterilizers. One company dealing with such applications is Asepsis, Inc. The steam sterilizer is predicated on rapid time, high efficiency principles with no destruction of internal components. Asepsis uses super-pure water to descale internal components (e.g. turbines). The high quality water is injected into the system during the sterilization cycle resulting in a clean, non-corrosive, short cycle machine. A 1:3 dilution of Solution A and a 1:5 dilution of Solution 5 were injected. In other words, final dilutions of 1:15 and 1:25 were made. What the attempt, in fact was, was to remove biofilm from the turbine which is involved in corrosion, resistance to sterilization and time required for the sterilization process.

The concept of cleaning and reduced time and efficiency of sterilization are absolutely key to the entire industry with world-wide applications. A time sequence was run to determine efficacy of the process with Solution A. The results are shown on a half cycle study.

| Sterilize | Time |
|---|---|
| Normal time for sterilization | 14 minutes |
| Solution A 1:3 dilution | 8 minutes |
| Solution 5 1:5 dilution | 10 minutes |

One million spores of Bacillus Stearothermophilus were inoculated onto the turbine along with an organic burden of 1% human serum and allowed to air-dry. This added organic and microbe load is considered to be the gold standard for disinfection and sterilization.

The results, generated after testing for microbial growth at 2, 4, 5, 6, 7, 8, 9, 10, 11 and 12 minutes, showed that Solutions A and 5 were capable of cleaning and aiding steam sterilization by reducing the time sequence one third to one half.

This is significant.

### Planktonic Cell Kill

Microbes were tested for planktonic cell kill. All listed below were rapidly killed using dilute Solution A and Solution 5, ATCC cultures or clinical isolates.
Bacillus stearothermophilus (high temperature spore former)
Bacillus subtilis (high and low temperature spore former)
Clostridium sporogenes (anaerobic spore former)
E. coli (including pathogenic species)
Pseudomonas aeuriginosa
Proteus vulgaris
Serratia marscens
Aerobacter aerogenes
Enterococci VRE
Streptococcus pyogenes
Staph epidermidis
Streptococcus group B
Staph aureus and MRSA
Strep pneumoniac
Mycobacteria phlei
Candida Albicans
Neisseria sicca

### Oral Microbes

Strep Mutans
Strep sanguis
Strep ruitis
Strep Salivarius
Bacteroides melanogenicus
Actinomyces odontolytius
Actinomyces israeli

### Cytotoxicity Studies

Numerous studies on animals showed no major or minor visual cytotoxic effects. The animals tested included New Zealand white rabbits and Guinea Pigs. Both deep and shallow wounds were inflicted and treated with Formula 5 by swabbing, foam applications, or by pouring over a wound. Known volumes of liquid were pipetted over wounds as well. Results showed no increase in healing time, hair regrowth, inflammation or scabbing; wound healing progressed very rapidly, exceeded Povidone Iodine treatment and phosphate buffered saline (PBS) pH 6.8 washes.

Studies using sprays into nostrils revealed no gross lung damage. Integration of 1:60 Formula 5 into the liquid diet of animals for five months revealed no organ damage in oral sites, larynx, pharynx, esophagus, stomach, small and large intestines, liver, spleen, rectum, or kidneys. Animals were capable of reproduction with no problems with offspring or numbers in a litter. Those offspring were capable of delivering normal offspring as well. Urine and feces were normal as well in output and appearance, no bleeding was ever detected.

I.V. and I.M. injections of 0.5 to 1.0 ml quantities of Formula 5 also showed no ill effects even when continued twice per day for six weeks. Tissue culture studies were performed on C3H/107½ mouse embryo cells. Very low cytotoxicity was noticed.

### Description of the Cell System Used to Study the Cytotoxicity and Genotoxicity of Formula 5

The cytotoxicity and genotoxicity of Formula 5, a novel antimicrobial agent was studied to determine whether Formula 5 exerted a uniquely high, intermediate, or low cytotoxicity to these murine cells. The testing system, we utilized the well-known cell culture system of C3H/10T1/2 CJ8 (10T1/2) mouse embryo fibroblasts. These cells are contact-inhibited, have a very low saturation density (approximately 800,000 cells/60 mm dish), and have a plating efficiency of approximately 25%-35%. The cells are grown in humidified incubators at 37° C. in an atmosphere of 5% carbon dioxide/air (v/v). 10T1/2 cells are thought to be a spontaneously immortalized, primitive mesenchymal cell line. These cells can be converted into adipocytes, myocytes and chrondrocytes when treated with the differentiation-inducing agent, 5-azacytidine. When treated with chemical carcinogens such as 3-methylcholanthrene, foci of transformed cells arise. When these foci are cloned and injected into nude mice, they form invasive progressively growing, fibrosarcomas.

### Progress Studying the Cytotoxicity of Formula 5

To determine how cytotoxic Formula 5 is to these non-transformed murine fibroblasts in cell culture cytotoxicity assays were conducted by standard methods in which 200 cells/60 mm dish, were plated five dishes per each concentration of Formula 5 tested. Formula 5 was added one day after the cells were seeded and remained in contact with the cells for 48 hours in the first set of cytotoxicity assays. Formula 5 was tested in a wide dilution series to determine the concentration ranges over which it was cytotoxic to 10T1/2 cells.

25 ul of Formula 5 were added to each cell culture dish containing 5 mls of medium that bathed the cells. In these experiments, 25 ul of each dilution of Formula 5 were added to 5 mls of cell culture medium bathing the cells. In the first cytotoxicity experiment, Formula 5 at a dilution of 1/2000 reduced the plating efficiency of cells to (92.8±3.4%) of the plating efficiency of control (phosphate buffered saline, PBS) treated cells. At a 1/200 dilution, Formula 5 caused the relative plating efficiency to actually increase to (109.6±10.2%), which was a slight hormetic effect that is often seen in these and other mammalian cells exposed to low levels of toxin. The molecular basis of this effect is not well understood. At a much higher concentration, a dilution of only 1/20, Formula 5 reduced the plating of the cells to (77.8±5.3%). At the very highest concentration of Formula 5 tested, a ½ dilution, the plating efficiency of the cells was reduced to 0% (all cells were killed). Hence, in this initial range-finding cytotoxicity experiment, the cytotoxicity of Formula 5 was dose-dependent at 1/20 and ½ dilutions. The concentrations of formula 5 were refined in successive experiments to be dilutions of 1/10 and lower.

In experiment 2, dilutions of 1/10, 1/100, 1/1,000 and 1/10,000 of Formula 5 against 10T1/2 cells were used. It was found that dilutions of Formula 5 of 1/10,000, then 1/1,000, then 1/100, then 1/10 caused a reduction in the plating efficiency of 10T1/2 cells to 93%, 85%, 95%, and then to 0% (assay 2). Hence, there was little or no cytotoxicity up to a dilution of 1/100, and then a precipitous decline in plating efficiency to 1% at a dilution 1/10 of Formula 5.

In a third assay, concentrations of a 1/20 dilution and a 1/10 dilution of Formula 5 were used and found that the plating of 10T1/2 cells was reduced to 65% and to 1.6% respectively.

In a fourth assay, test concentrations of Formula 5 of 1/20 and greater were used. In this experiment, a concentration of 1/20 of Formula 5 reduced the plating efficiency of 10T1/2 cells to 62.8%. It was found that concentrations of 2/20, 3/20, 4/20, 5/20 and higher than this killed all the 10T1/2 cells; the plating efficiency of treated cells divided by that of control cells was 0%. Hence, the cytotoxicity of Formula 5 was clearly dose-dependent and occurred at concentrations greater than 1/20 of Formula 5. This experiment was repeated exactly in a new experiment (#5), and found that a 1/20 concentration of Formula 5 reduced the cytotoxicity of 10T1/2 cells to 74.7%, and that a concentration of 2/20 dilution of Formula 5 and higher concentrations reduced the plating of 10T1/2 cells to 0%. Hence, the data in experiments 4 and 5 were fairly consistent.

In experiment #6, concentrations of Formula 5 flanking the 1/20 dilution, both higher and lower concentrations, to define a cytotoxicity curve, were used. In this experiment, concentrations of 1/100 dilution, 1/50, 1/33.3, 1/25, 1/20 and 1/10 dilutions caused reductions in the plating efficiency of 10T1/2 cells to 93%, 85.4%, 83.3% and 67.4%.

Next this data was cumulated in tabular form and averaged from the results of all the experiments. As shown in this table, when the results so fall the experiments are averaged, concentrations of 1/10,000, 1/2,000, 1/1,000 and 1/200 cause little or no cytotoxicity. At concentrations of 1/100, 1/50, 1/33.3, 1/25, 1/20, 1/10 and 3/20, there are reductions in the plating efficiency of 10T1/2 cells to 94%, 85.4%, 83.3%, 67.7%, 74.0%, 16.1% and 0% respectively. Hence, the cytotoxicity of Formula 5 is dose-dependent in this concentration range. The LC50 value (concentration that reduces the plating efficiency to 50% of that of control cells), is estimated to be between a 1/25 and a 1/10 concentration of Formula 5. After completing these experiments, information was received that the concentration of the Formula 5 solution provided was 63.5 mg/ml of solids. Hence, it was estimated that the actual LC50 value was: 12.7 ug/ml<LC50<31.8 ug/ml. Formula 5 is certainly not as cytotoxic as adriamycin, whose LC50 is 0.0158 ug/ml, nor as cytotoxic as the metabolite of the fungus Aspergillus, whose LC50 value is 1.50 ug/ml. The LC50 value of Formula 5, between 13 and 32 ug/ml, ranks it slightly above the carcinogenic polycyclic aromatic hydrocarbon, 3-methylcholanthrene. Similarly, Formula 5 is 5 and 70 times more cytotoxic to 10T1/2 cells than acetaminophen, aspirin and borax, whose LC50 values are 1,000 ug/ml, 1,500 ug/ml and 2,000 ug/ml, respectively. Hence, Formula 5 has intermediate cytotoxicity as constituted.

Conclusions

Therefore, the order of cytotoxicity (LC50 values in parentheses), was: Formula 5 (22 ug/1 ml)>acetaminophen (tylenol, 500 ug/mll)>phenacetin (1,000 ug/ml)>aspirin (1,500 ug/ml). A comparison of the highest non-cytotoxic doses (in parentheses) of these compounds yields the exact same order of cytotoxicity: Formula 5 (0.002 mg/1 ml)>acetaminophen (0.05 mg/ml)>phenacetin (0.18 mg/ml)>aspirin (0.25 mg/ml).

TABLE 2
CYTOTOXICITY OF VARIOUS CHEMICALS TO 10T1/2 CELLS

| | | | | Highest Non- |
|---|---|---|---|---|
| | | | | Cytotoxic |
| Chemical | LC50 value, ug/ml | | LC50 value, uM | Concentration |
| Adriamycin | 0.0158 | ug/ml | 0.03 | |
| BaP-anti-diol | | | | |
| epoxide | 0.0755 | ug/ml | 0.25 | |
| Flubendazole | 0.15 | ug/ml | | |
| Aflatoxin B1 | 1.50 | ug/ml | | |
| Benzo(a)pyrene | 3.78 | ug/ml | 15.0 | |
| N-acetoxy-acetyl | 3.99 | ug/ml | 15.0 | |
| aminofluorene | | | | |
| 3-methylcholanthrene | 10.74 | ug/ml | 40.0 | |
| Formula 5 | 12.7-31.8 | ug/ml | | 0.002 mg/ml |
| | | | | (approximate) |
| Ouabain | 500 | ug/ml | | |
| Acetaminophen | 500 | ug/ml | | 0.05 mg/ml |
| | | | | (approximate) |
| Phenacetin | 1,000 | ug/ml | | 0.18 mg/ml |
| | | | | (approximate) |
| Aspirin | 1,500 | ug/ml | | 0.25 mg/ml |
| | | | | (approximate) |
| Refined Borax | 2,000 ± 1,200 | ug/ml | | |

In a delicate cell culture system there is slight toxicity at high concentrations whereas virtually none is detected in other cell cultures nor in animals.

| Plant | Time | | Area Sprayed | Results |
|---|---|---|---|---|
| Hydrangea | 2 | days | Flower | Browning |
| | | | Leaves | Drying |
| Rose | 2-3 | days | Petals | Browning |
| Gladiolas | 2-3 | days | Flower | Dead |
| Palm | 2-3 | weeks | Fronds | White spots |
| Hibiscus | 3-5 | days | Flowers | Dead |
| | | | Leaves | Drying |
| Figs | 3 | days | Leaves | Dead |
| Peach | 2 | weeks | Fruit, leaves | Slight effect |
| Succulents | 3 | weeks | Arid succulents | No effect |
| Rosemary | 1 | week | Leaves | Drying or dead |
| | | | | Starting to die |
| Mint | 1 | week | Leaves | Drying or dead |
| | | | | Starting to die |
| Thyme | 2 | days | Leaves | Falling off |
| Tomatoes | 3 | days | Leaves, blossoms | Dead |
| Camellias | 2 | weeks | Leaves | Slight effect |
| Ants | 1 | minute | | Dead |
| Spiders | 1 | minute | | Dead |

### Horticultural Studies

A probe was made to ascertain whether Solution A had properties of value in the plant world. A 1:20 dilution was made and put in spray bottles. Plants were sprayed daily with a few misting sprays and observed. A variety of flowers, herbs, plants and insects were put into test.

| Plant | Time | | Area Sprayed | Results |
|---|---|---|---|---|
| Grapes | 3 | days | Young growing tip | Killed day 1 |
| | 3 | days | Young growing ½ leaf | Dying day 2 |
| | 3 | days | Older mature leaf | Affected day 3 |
| | 3 | days | Green grapes | Bottoms turn black |
| | 2 | weeks | Back of old wood | No effect on bark |
| | | | | or vine |

Conclusion

For the most part Solution A is toxic to plants.

### Temperature Effects on the Microbiocidal Activity of Chemical Formulations Based Compositions (Formula A and 5)

The brunt of studies have been performed at room temperature (ambient) at 70° F. and a humidity ranging from 20-60%. Temperature range varied from 55° F. to 90° F.

At temperatures, ranging from 38° to 48° F. precipitates of glycerol complexes are noted which dissipate upon warning to 50° F.

As many of the uses of these compounds will be used on surface skin wounds of varying depths, utilization of warm mixtures at higher temperatures are inappropriate owing to the burning action of the heated liquid.

Studies at varying temperatures were performed on single cell (planktonic) microbial populations, on mixed cultures of numerous combinations of microbes on biofilms were generated in silicon tubing as foravages, or catheter implants. Mixed cultures of a heterotypic population either in suspension culture or in a biofilm mixture were also tested.

Stock bacterial cultures were grown in 100 ml bottles of Trypticase Soy Broth, Nutrient Broth, Yeast Extract and Todd-Hewitt Broth (2:1:1:1) Formulas 5 and A were supplied as a 1:5 solution and were dilute 2 fold down to 1:256, A 0.5 ml aliquot of the various dilution series were added to 2.0 ml of media containing 36 hour cultures make up to a final bacterial concentration of 1 million to 100 million bacteria per ml.

At various time points e.g. 10, 20, 30, 40 and 50, seconds up to 30 minutes 0.5 ml of the test solution was pipetted into a Petri dish and 12 ml media with 1.2% agar added as a hardening agent and the bacteria, media antimicrobial mixture was made into a pour plate for a colony count (CFU). After 48-72 hours, the colonies were counted and recorded.

Results indicated that the ambient temperature worked very affectedly on killing bacteria whether they were in a biofilm or Planktonic configuration. Heating served no real purpose.

Figure one shows a composite table of the results showing the high level of microbial kill at all temperature ranges.

In studies utilizing rabbits and guinea pigs in wounding studies, the animal's body temperature had no effects. When animal wounds were treated with Formula A or 5 no increase in body temperature (fever) was noted.

### Biofilms

Biofilms are composed of masses of interactive bacteria usually attached to a solid surface. The surface may be rocks in a streambed providing a slippery carpet. A biofilm may also attach to pipes in a waterline or in an industrial plumbing system and wreak slow or rapid havoc. When a biofilm is associated with medical problems, it may attach to catheter tubing causing infections or to bone resulting in major problems in implants of bone or hip replacements or even in heart valves.

One of the many major problems causing distress is the ability of biofilms to cause resistance to antibiotics or other drugs. When microbes first increase in numbers, they emit chemicals, which cause a gathering into colonies in large numbers; a process known as quorum sensing. Other chemical triggers allow for “road formation” between “high rise colonies” which allow for transport of waste materials and simultaneous transport of food stuffs ad oxygen. Other chemical signals trigger enzymes, causing antibiotic destruction or numerous other functional molecules involved in the pathogenesis process. Microbes start behaving like multicellular organs and they are difficult to destroy and inhibit. There are few reports about the destruction of microbes without host damage or the dispersion of microbes and with the subsequent continuous destruction of the resultant planktonic single cells.

The dispersion and destruction of just such a mechanism of microbial breakdown in not only single hemotypic populations, but in multiple species interactive biofilms (heterotypic) as well.

### Biofilm Formation in the Lab

Silicon based catheter or lavage tubes were filled with media containing the test organism(s). In some cases, single homogeneous species were used, e.g. E. coli, Pseudomonas or Candida. In other instances, combinations such as E. coli and Pseudomonas were used 1:1. In other instances, E. Coli and Candida were used in two mix populations of heterogeneous population, whereas in other tests, three species were mixed. In the most complex test, a mixed culture of oral microbes was used mixing perhaps hundreds of species. Formula 5 was capable of breakdown of all biofilms and subsequent destruction of the detached microbes. Cultures containing Pseudomonas and Candida species were roost resistant taking 15-30 minutes for complete kill. Most other species were destroyed within five minutes.

The action of Formula 5 started as a slow removal of the biofilm mass in large pieces from silicon tubing or glass surfaces. This appeared analogous to vitronection destruction by EDTA mammalian cell cultures. In those cases, mammalian cells were removed in large fragments resembling tissue paper. Later, a second action of cell to cell adherence set in and the proteinaceous adherence material was broken by Trypsin resulting in a single cell suspension.

In the case of bacteria, a two-phase breakdown of adherence (to a solid surface) followed by a breakdown of coherence (cell to cell adhesion) then taking place. When one only induces or treats to breakdown adherence to a surface the one disseminates viable cell clots to new areas to enhance the biofilm formation process.

Destruction of cells moves into a solubilization stage where the cells shrivel up and finally burst. Whole centrifuged (5000×g) cell masses have been pelleted, washed with saline twice, resuspended and placed in fresh, new media or filtered, conditioned media in which cells had formally been grown. In no instance were any new cells grown or detected even after 15 days incubation.

To ensure that bacteria had a constant supply of fresh media, a peristaltic pump with a number 18 gauge needle attached to tubing at the end of the pump and tubing systems was used. The flow rate was 1 ml/10 minutes. Biofilms were formed over a three day, five day and seven day period. Tubing was cut in lengthwise strips, washed in saline and fixed in gluteraldehyde, metal sprayed and examined in a Cambridge scanning electron microscope at 1,000 to 8,000 magnifications. Note the data shown in the Figures identified below:

FIG. 16: A solid biofilm mass on zero time five day formation.

FIG. 15: A seven minute treatment of Formula 5. The biofilm mass is shriveling down and detaching.

FIG. 22: Mixed oral culture coated with a gelatinous mass coating.

FIG. 21: Reveals a five minute exposure to Formula 5 where the mass is disintegrated.
No viable cells could be detected after 15 days growth, even in pellet debris or by feeding media to the test tube remnants.
   Formula A and 5 and their Effect on Sperm Motility with the Potential of a Spermicide and a Fertility Blocker

The number one cause of male infertility is the loss of sperm motility. The cessation, or even slowing down of sperm movement driven by flagellar action, is an essential element in the fertilization of most all living creatures. Males with a number of sperm reaching 100 million, but lacking in motility, or even having poor motility, are generally lacking in the capacity for locating the ovum and egg penetration. Spermicides capable of inducing sperm immobility are effective birth control regulators.

This report discusses the excellent capacity on sperm motility reduction and elimination in a rapid time period. These formulations are capable of being applied vaginally as sprays, gels, creams and other delivery systems, to rapidly and effectively eliminate sperm motility and hence fertilization capability.

Sperm was obtained from healthy male donors in their early to mid-twenties at the height of their reproduction years. All ejaculates exceeded 3 mls and were in the range of 95% or higher in motility. Five subjects were accepted in the study program.

Ejaculates were diluted in PBS saline pH 6.8 at ambient temperature (70° F.). Controls without formulations added, except PBS saline, were counted every 10 minutes for up to four hours. Test samples had 10 microliters to 1.00 microliters of Formula added to 1 ml of sperm in PBS containing up to 25 million sperm. This mixture was added to baemocytometer slides and sample areas were counted by standard methods of counting to determine sperm motility.

In less than one minute, and often in 10-30 seconds, depending on the concentration, motility was eliminated. At times, sperm would spin without directional movements for a number of seconds, but the inhibition was rapid and completely effective.

Controls were capable of viability ad motility for hours with effective ranges of viability from 40-85% even after several hours. These solutions provide a solid basis for regulation of fertility in addition to having antimicrobial activity.

### The Effects of Formula A and 5 on Vaginal Tissue and the Potential as an Antimicrobial as a Treatment for Venereal Disease and Candidasis

There are in excess of 20 microbes capable of causing venereal diseases, syphilis, gonorrhea, genital warts, herpes, chlamydia and aids are prevalent in the world today. Vaginal itch or candidacies of the vaginal area affect about 20 million women in the USA every summer.

Obviously, the associated costs, suffering and treatment is enormous. Initial studies were in New Zealand white rabbits to test of cytotoxil or inflammatory effects on Solution A and 5.

Five female rabbits at 3 months of age were swabbed intravaginally with the test solution twice a day for 7 days. Swabs were made on day one zero time and colony-forming units were determined.

Nutrient Agar+1% Tryicase Soy Agar were used for test media, as were blood Agar plates. The animals were examined twice daily for inflammation or lesion formation.

Results showed a major reduction in normal vaginal flora, at least 75%. When swabbing was completed often 7 days, the flora returned to original CFU'S within 48 hours.

No redness, irritation, or lesion formation was detected by visual examination. Furthermore, the rabbits showed no signs of discomfort or irritation and behaved perfectly normal during treatment and the entire 7 day period.

Formulations can be compounded into creams, sprays and ointments for topical applications.

### Utilization of Betaine Compounds for the Prevention of Food Spoilage

A major concern in today's world is the spoilage of fresh foods and meats and the use of bioterrorism to destroy food products and disrupt the infrastructure of nations. Certain foods such as poultry and pork have limited shelf lives wing to the microbes indigenous to their skin surfaces. Salmonella and Shigella are common on poultry and grow rapidly. In a matter of a few days pork can go rancid even under refrigerated conditions when temperatures slightly rise. One can feel the slime layers building up as a slippery second skin and the associated odors are putrid. The growth rates of bacteria are extraordinary especially when the foodstuffs have a high initial seeding of microbes and are not properly refrigerated, frozen or packed. Our shipping via the trucking industry is long distance and time consuming, adding to the problem. The microbial population can be indigenous and native flora, or seed from human sources. The studies presented here were incubated at lower temperatures entitled room temperature which range from 40-60° F. or at incubator temperature, 37° C. (98° F.).

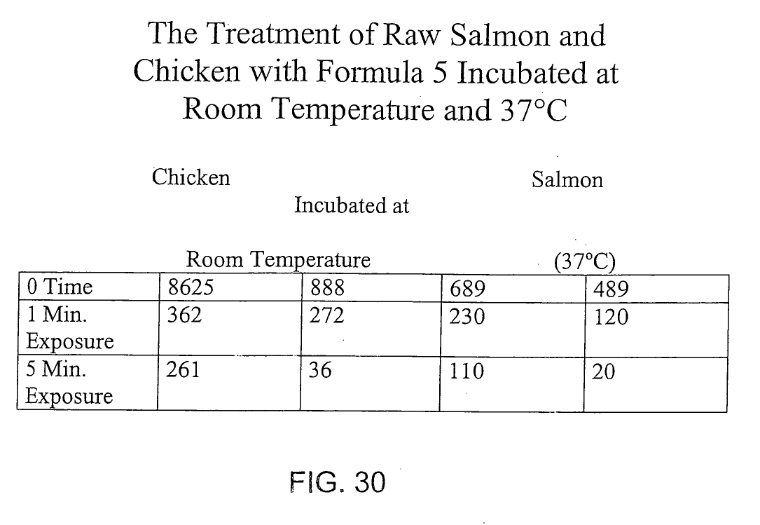

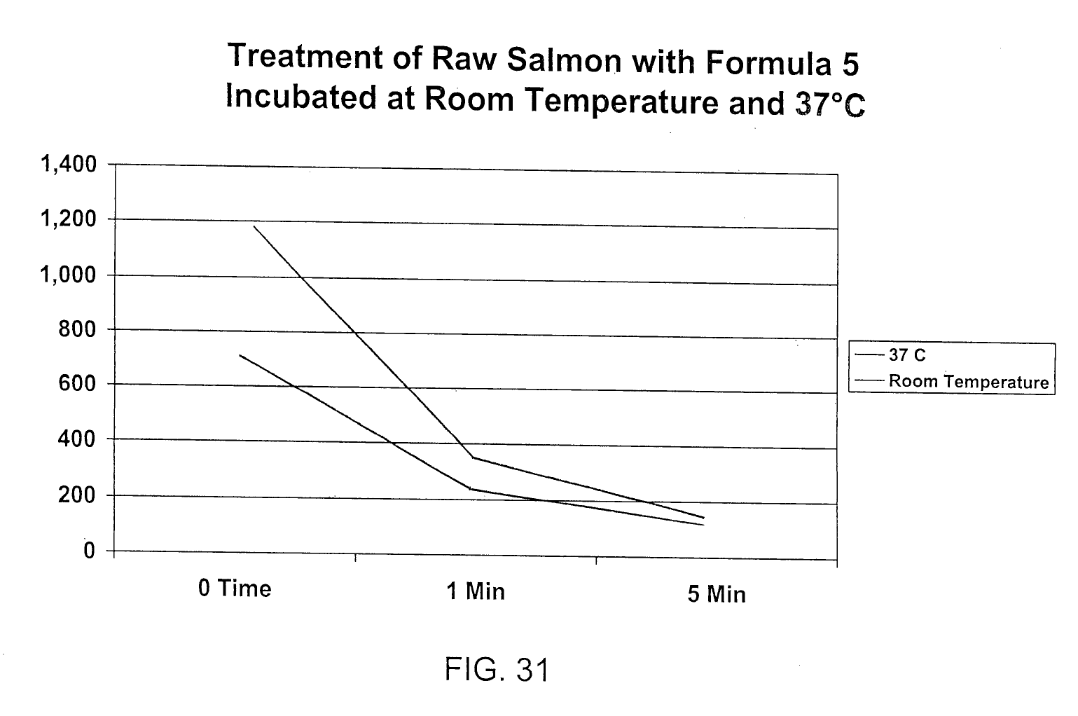

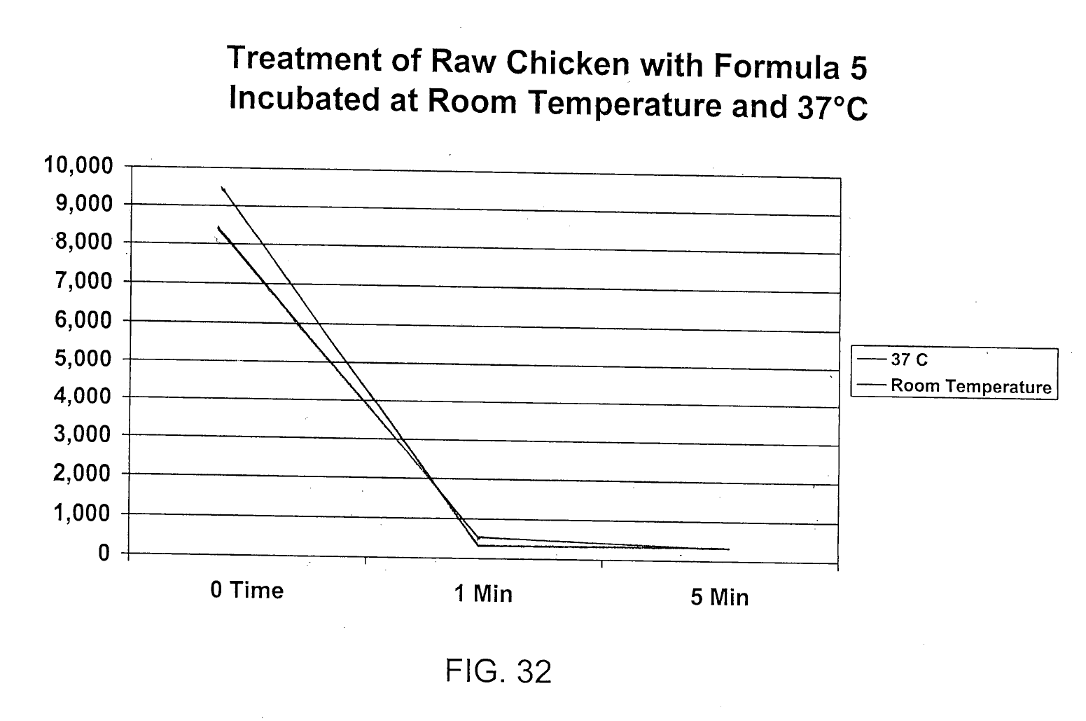

The results are shown in FIGS. 30-32 following fresh wild salmon (Keta) from Alaska, were purchased at a local market as were cut up chicken pieces (Foster Farm). Sterile cotton swabs were used to wipe the surfaces of the two specimen foods. The swabs were used to streak blood agar plates in triplicate. They were called 0 time starting cultures; one set was used for incubation at one temperature (room temperature) and one at 37° C. in an incubator. Other blood agar plates were done in triplicate for one minute after swabbing the same as before and followed by swabbing with Formula 5. Results showed that more colony forming units (CFU's) were detected at ambient temperatures than in the incubator. This is not unsuspected as indigenous chicken skins and fish would have flora accustomed to growing at lower temperatures, especially when in cold storage. In all cases, 0 time start was reduced significantly in 1 minute exposure to Formula 5. This reduction in numbers continued after 5 minutes as well. Formula 5 could be applied by spray, total immersion, or other means of application. Times of exposure could be extended to increase microbial kill even longer than that of five minutes.

Other notable observations include no skin or flesh discoloration, no blemishing, no skin destruction, no alteration in taste or odor after 48 hours of storage or after cooking. All of these observations point to a very good approach to extending shelf life and safety for ingestion and the potential for detoxing.

The tests presented above which expressly refer only to Solution A were repeated with Solution 5, with equal or somewhat better results.

### The Formulas

One version of Formula A comprises so alcohol (typically 8% by weight), glycerin (typically 6% by weight), amine oxide (typically up to 1% by weight), methyl betadine (typically 0.3% by weight) and betadine NaF (typically 0.02% by weight).

A second version of Formula A comprises an admixture of cetyldimethylbetaine, lauryldimethylbetaine, myristyldimethylbetaine, myristyldimethyl amine oxide, and cetyldimethyl amine oxide.

The basic composition of Formula 5 is myristyldimethylbetaine, within the range of 0.20 to 2% by weight, cetyldimethyl amine oxide, within the range of 1.0 to 2.5% by weight, and citric acid sufficient to place the pH of the within a pH range of 3.5 to 6.5%. The addition of lauryldimethylbetaine, within the range of 0.4 to 2.0% by weight, enhances the composition.

Cocodimethulbetaine, within the range of 0.4 to 2.0% by weight, may be substituted for laurylbetaine, but is less effective.

Cocodimethyl amine oxide, within the range of 1.0 to 2.5% by weight, may be substituted for cetyldimethyl amine oxide but is not as effective.

### ADVANTAGES OF THE PRESENT INVENTION

The material is capable of rapid kill of literally all microbes tested, be they resistant spores, gram positive, gram negative, rods, cocci, spirillum or yeast cells.

Its action on membranes and bacteria make it a natural for virucidal activity.

For all of Formula 5's antimicrobial activity it has very low cytotoxic action with the exception on New Zealand white rabbit eye (albino) which can redden with water. This only happens with fully concentrated Formula (5%).

Can be ingested or injected. Formula 5 is not inactivated to any significant level (ca. 8%) by organic matter such as blood serum and skin secretions.

The Formula is spermicidal and is not toxic to vaginal tissue. Formula 5 had the potential to be a killer of STD cells, including gonorrhea, syphilis, Chlamydia, genital warts and HIV and also be a spermicide.

It may be antimicrobial eyewash when used at concentrations diluted to 1:60 or further.

Has the potential to treat pneumonia.

Effective on wounds both prophylactically and therapeutically.

Effective on burns both prophylactically and therapeutically.

Effective on animal lesions, hoof rot and white line disease.

Effective as a shampoo, wash, soap and surface disinfectant.

Destroys biofilms and has the potential to clean joint replacements, catheter infections, kidney and bladder infections without tissue damage.

The invention may be embodied in other specific forms without departing from the spirit of essential characteristics thereof. The present embodiments therefore to be considered in all resects as illustrative and are not restrictive, the scope of the invention being indicated by the appended claims rather than by the foregoing description, and all changes which come within the meaning and range of equivalency of the claims are therefore intended to be embraced therein.

What is claimed is:

1. A method of alleviating microbials comprising the acts of:
formulating an admix composition of:
(a) a mixture of two alkyl-N-betaines,
(b) an alkyl-N,N-dimethylamine oxide, and
(c) a protonating agent in an amount sufficient to adjust the pH of the overall composition in the range of from about 4 to about 7.5.
causing the composition to be applied to the location inhabited by microbials to reduce the count thereof.

2. A method according to claim 1 wherein the protonating agent is selected from the group consisting of hydrochloric acid.

3. A method according to claim 1 wherein the location is an object where the microbials comprise biofilm.

4. A method according to claim 1 wherein the causing act occurs at non-elevated ambient temperatures.

5. A method of alleviating microbials comprising the acts of:
formulating an admix composition comprising:
myristyldimethylbetaine;
cetyldimethyl amine oxide; and
citric acid in an amount sufficient to adjust the pH of the overall composition within the range of 3.5 to 6.5.
causing the composition to be applied to a location inhabited by microbials to reduce the count thereof.

6. A method according to claim 5 wherein the location is an object where the microbials comprise biofilm.

7. A method according to claim 5 wherein the causing act occurs at non-elevated ambient temperatures.

8. A method according to claim 5 wherein the myristyldimethylbetaine is within the range of 0.2 to 2.0% by weight.

9. A method according to claim 5 wherein the cetyldimethyl amine oxide is within the range of 1.0 to 2.5% by weight.

10. A method according to claim 5 wherein the composition further comprises lauryldimethylbetaine within the range of 0.4 to 2.0% by weight.

11. A method according to claim 10 comprising the act of substituting cocodimethylbetaine, within the range of 0.4 to 2.0% by weight, for the laurylbetaine.

12. A method according to claim 10 comprising the act of substituting cocodimethyl amine oxide, within the range of 1.0 to 2.5%, for the cetyldimethyl amine oxide.
